SEQUENCE LISTING (56 sequences)

SEQ ID NO: 1 - DNA, 195 bp, Artificial Sequence
  mRNA BINDING MOTIF
ctcagtgcagcaatccaccgcacccaactctggttccacgggcgcatttcccgtgaggagagccagcggcttattggacagcagggcttggtagacggcctgttcctggtccgggagagtcagcggaacccccagggctttgtcctctctttgtgccacctgcagaaagtgaagcattatctcatcctgccgagc

SEQ ID NO: 2 - protein, 65 aa, Artificial Sequence
  AMINO ACID SEQUENCE OF AN mRNA BINDING MOTIF
LSAAIHRTQLWFHGRISREESQRLIGQQGLVDGLFLVRESQRNPQGFVLSLCHLQKVKHYLILPS

SEQ ID NO: 3 - DNA, 195 bp, Artificial Sequence
  ANTISENSE SEQUENCE OF AN mRNA BINDING MOTIF
gctcggcaggatgagataatgcttcactttctgcaggtggcacaaagagaggacaaagccctgggggttccgctgactctcccggaccaggaacaggccgtctaccaagccctgctgtccaataagccgctggctctcctcacgggaaatgcgcccgtggaaccagagttgggtgcggtggattgctgcactgag

SEQ ID NO: 4 - DNA, 259 bp, Artificial Sequence
  A NOVEL 259 bp AURE
ccgactagccaggaagtacttccacctcgggcacattttgggaagttgcattcctttgtcttcaaactgtgaagcatttacagaaacgcatccagcaagaatattgtccctttgagcagaaatttatctttcaaagaggtatatttgaaaaaaaaaaaaaaagtatatgtgaggatttttattgattggggatcttggagtttttcattgtcgctattgatttttacttcaatgggctcttccaacaaggaagaagctt

SEQ ID NO: 5 - DNA, 73 bp, Artificial Sequence
  Synthetic
aaactgtgaagcatttacagaaacgcatccagcaagaatattgtccctttgagcagaaatttatctttcaaag

SEQ ID NO: 6 - DNA, 73 bp, Artificial Sequence
  Synthetic
gtatatgtgaggatttttattgattggggatcttggagtttttcattgtcgctattgatttttacttcatggg

SEQ ID NO: 7 - DNA, 34 bp, Artificial Sequence
  Synthetic
gtatatgtgaggatttttattgattggggatctt

SEQ ID NO: 8 - DNA, 40 bp, Artificial Sequence
  Synthetic
ggagtttttcattgtcgctattgatttttacttcaatggg

SEQ ID NO: 9 - DNA, 34 bp, Artificial Sequence
  Synthetic
gtatatgtgaggatgggtattgattggggatctt

SEQ ID NO: 10 - DNA, 40 bp, Artificial Sequence
  Synthetic
ggagtttttcattgtcgctattgatgggtacttcaatggg

SEQ ID NO: 11 - DNA, 29 bp, Artificial Sequence
  Synthetic
ttttactttgttatttattattttatata

SEQ ID NO: 12 - DNA, 20 bp, Artificial Sequence
  Synthetic
gtatatgtgaggatttttat

SEQ ID NO: 13 - DNA, 20 bp, Artificial Sequence
  Synthetic
ataaaaatcctcacatatac

SEQ ID NO: 14 - DNA, 14 bp, Artificial Sequence
  Synthetic
tgattggggatctt

SEQ ID NO: 15 - DNA, 14 bp, Artificial Sequence
  Synthetic
aagatccccaatca

SEQ ID NO: 16 - DNA, 12 bp, Artificial Sequence
  Synthetic
gtatatgtgagg

SEQ ID NO: 17 - DNA, 12 bp, Artificial Sequence
  Synthetic
cctcacatatac

SEQ ID NO: 18 - DNA, 18 bp, Artificial Sequence
  Synthetic
ggagtttttcattgtcgc

SEQ ID NO: 19 - DNA, 18 bp, Artificial Sequence
  Synthetic
gcgacaatgaaaaactcc

SEQ ID NO: 20 - DNA, 22 bp, Artificial Sequence
  Synthetic
tattgatttttacttcaatggg

SEQ ID NO: 21 - DNA, 22 bp, Artificial Sequence
  Synthetic
cccattgaagtaaaaatcaata

SEQ ID NO: 22 - protein, 55 aa, Artificial Sequence
  Synthetic
WFHHKISRDEAQRLIIQQGLVDGVFLVRDSQSNPKTFVLSMSHGQKIKHFQIIPV

SEQ ID NO: 23 - protein, 55 aa, Artificial Sequence
  Synthetic
WFHGRISREESHRIIKQQGLVDGLFLVRDSQSNPKAFVLTLCHHQKIKNFQILPC

SEQ ID NO: 24 - protein, 55 aa, Artificial Sequence
  Synthetic
WFHGRISREESQRLIGQQGLVDGLFLVRESQRNPQGFVLSLCHLQKVKHYLILPS

SEQ ID NO: 25 - protein, 55 aa, Artificial Sequence
  Synthetic
WFHLRLSREESQRLIGKGGLVLGLLLVRESQRIPLGFVLSLCHLQKIKIYGILPS

SEQ ID NO: 26 - protein, 65 aa, Artificial Sequence; identical to SEQ ID NO: 2
  Synthetic

SEQ ID NO: 27 - protein, 21 aa, Homo Sapiens
VSREESHRIIKQQGLVDGLFL

SEQ ID NO: 28 - protein, 21 aa, Homo Sapiens
VSREESHRIIKQQGLVDGLFL

SEQ ID NO: 29 - protein, 21 aa, Homo Sapiens
VSREESHRIIKQQGLVDGLFL

SEQ ID NO: 30 - protein, 21 aa, Mus Musculus
VSREESHRIIKQQGLVDGLFL

SEQ ID NO: 31 - protein, 21 aa, Homo Sapiens
VSREESHRIIKQQGLVDGLFL

SEQ ID NO: 32 - protein, 21 aa, Homo Sapiens
VSREESHRIIKQQGLVDGLFL

SEQ ID NO: 33 - protein, 21 aa, Homo Sapiens
VSREESHRIIKQQGLVDGLFL

SEQ ID NO: 34 - protein, 21 aa, Mus Musculus
VSREESHRIIKQQGLVDGLFL

SEQ ID NO: 35 - protein, 21 aa, Mus Musculus
VSREESHRIIKQQGLVDGLFL

SEQ ID NO: 36 - protein, 21 aa, Mus Musculus
VSREESHRIIKQQGLVDGLFL

SEQ ID NO: 37 - protein, 21 aa, Mus Musculus
VSREESHRIIKQQGLVDGLFL

SEQ ID NO: 38 - protein, 21 aa, Homo Sapiens
ISREESHRIIKQQGTPRALSS

SEQ ID NO: 39 - protein, 21 aa, Homo Sapiens
ISREESQRLIGQQGLVDGLFL

SEQ ID NO: 40 - protein, 21 aa, Homo Sapiens
ISREESQRLIGQQGLVDGLFL

SEQ ID NO: 41 - protein, 21 aa, Homo Sapiens
ISREESQRLIGQQGLVDGLFL

SEQ ID NO: 42 - protein, 21 aa, Mus Musculus
ISREESQRLIGQQGLVDGVFL

SEQ ID NO: 43 - protein, 21 aa, Mus Musculus
ISREESQRLIGQQGLVDGVFL

SEQ ID NO: 44 - protein, 21 aa, Rattus Norvegicus
ISREESQRLIGQQGLVDGVFL

SEQ ID NO: 45 - protein, 21 aa, Homo Sapiens
ISRDEAQRLIIQQGLVDGVFL

SEQ ID NO: 46 - protein, 21 aa, Homo Sapiens
ISRDEAQRLIIQQGLVDGVFL

SEQ ID NO: 47 - protein, 21 aa, Homo Sapiens
ISRDEAQRLIIQQGLVDGVFL

SEQ ID NO: 48 - protein, 21 aa, Rattus Norvegicus
ISRDEAQQLITRQGPVDGVFL

SEQ ID NO: 49 - protein, 21 aa, Zea Mays
ISSLPTLVLIGRQGPVDGKTL

SEQ ID NO: 50 - protein, 7 aa, Artificial Sequence
  Synthetic
IIIGGII

SEQ ID NO: 51 - protein, 4 aa, Artificial Sequence
  Synthetic
LLLL

SEQ ID NO: 52 - protein, 3 aa, Artificial Sequence
  Synthetic
VVV

SEQ ID NO: 53 - protein, 22 aa, Artificial Sequence
  Synthetic
RISREESQRLIGQQGLVDGLFL

SEQ ID NO: 54 - DNA, 69 bp, Homo Sapiens
tttgtacagagtgcttttctgtttagtttttactttttttgttttgtttttttaaagacgaaataaaga

SEQ ID NO: 55 - DNA, 73 bp, Artificial Sequence; identical to SEQ ID NO: 6
  Synthetic

SEQ ID NO: 56 - DNA, 73 bp, Artificial Sequence
  Synthetic
gtatatgtgaggatttttagtgcttggttctgtttagtttttactttttttgttttgtttttttaaagacgag

(19) United States
(12) Patent Application Publication
(10) Pub. No.: US 2004/0121323 A1
(43) Pub. Date: Jun. 24, 2004

(54) Mrna binding motif

(76) Inventors: Peter J. Leedman, Mount Claremont (AU); Lois Balmer, Armadale (AU); Andrew Thomson, Perth (AU)

Correspondence Address: WILLIAMS, MORGAN & AMERSON, P.C., 10333 RICHMOND, SUITE 1100, HOUSTON, TX, 77042, US

(21) Appl. No.: 10/168,781

(22) PCT Filed: Dec. 22, 2000
(86) PCT No.: PCT/AU00/01595

(30) Foreign Application Priority Data
Dec. 23, 1999 (AU) PQ4835

(51) Int. Cl. C12Q 1/68; C07H 21/04

(52) U.S. Cl. 435/6; 536/23.1

(57) ABSTRACT

This application is concerned with a messenger ribonucleic acid (mRNA) binding motif. It is particularly concerned with a mRNA binding motif that is capable of binding and destabilizing the mRNA. This provides for an mRNA binding motif having: a) a nucleotide sequence as shown in SEQ ID NO:1; or b) a biologically active fragment of the sequence in a); or c) a nucleic acid molecule which has at least 75% sequence homology to the sequences in a) or b); or d) a nucleic acid molecule which is capable of hybridizing to any one of the sequences in a) or b) under stringent conditions.

The present application is a United States nationalization of PCT International Patent Application PCT/AU00/01595, filed Dec. 22, 2000, which claims priority to Australian Patent Application Serial No. PQ4835, filed Dec. 23, 1999.

### FIELD OF THE INVENTION

This application is concerned with a messenger ribonucleic acid (mRNA) binding motif. It is particularly concerned with a mRNA binding motif that is capable of binding and destabilizing the mRNA. This application also relates to methods of screening for molecules that bind to the mRNA binding motif, and methods of affecting the activity of the mRNA binding motif in vivo.

### BACKGROUND OF THE INVENTION

Tyrosine kinase receptors are involved in the regulation of a number of important cellular activities including, the regulation of cellular growth, differentiation, motility and metabolism. These receptors bind specific hormones and growth factors, thereby activating specific signalling pathways. Hence, these receptors have a central role in one of the most important intracellular communication pathways.

One sub-population of the tyrosine kinase receptor family is the epidermal growth factor receptor (EGF-R) family. There is now evidence that the EGF-R plays a central role in the pathogenesis of malignancy. The EGF-R appears to especially have a role in the growth and proliferation of multiple human cancers, including breast, prostate, skin, colon, bladder and brain. The EGF-R has been shown to be overexpressed (Yamamoto et al., 1986) and/or amplified (Filmus et al., 1985; Merlino et al., 1984) in various human tumour cell lines, including the MDA-468 and BT-20 breast, and the A431 and KB epidermoid carcinoma cell lines. It has also been shown that the over-expression of a structural and functional homologue of EGF, TGF-α, in transgenic mice produces mammary tumours (Sandgren et al., 1990). It was noted in the early 1990's that the expression of the EGF-R proto-oncogene directly influences the pathogenesis of human breast cancer (Fernandez-Pol, 1991). Indeed, the level of EGF-R expression has proven clinically useful, as it is higher in hormone-independent breast cancers, which are usually estrogen receptor negative (ER-) (Klijn et al., 1993). Further, high levels of EGF-R expression in breast cancer biopsies correlates with a poor prognosis and the poorest response to first line hormone treatment, independent of ER status (Klijn et al., 1993).

Accordingly, as will be appreciated by those skilled in the art, there is a need to understand the molecular mechanisms governing the control of EGF-R expression in cancer cells, as this may have important implications in the design of effective means of treating certain cancers.

Present State of Knowledge of the Molecular Mechanisms

The EGF-R family of tyrosine kinase receptors contains 3 structurally related members, erbB-2, erbB-3 and erbB-4. All of these members share a common overall architecture, consisting of an N-terminal region containing a highly conserved proline-rich decapeptide motif, a central region harbouring a Pleckstrin homology (PH) domain, and a C-terminal Src homology 2 (SH2) domain.

erbB-2 is frequently amplified up to 30% in human breast, and ovarian cancers, and there is a direct correlation between the level of amplification and expression with clinical outcome (Salamon et al., 1989). It has been noted that all of the erbB's have some association with heregulins (HRGs). HRGs are TGF-α-related ligands that were originally thought to bind to erbB-2 (Holmes et al., 1992). However, HRGs bind to, erbB-3 and erbB-4. erbB-2 indirectly participates in HRG-mediated signaling through transphosphorylation, or receptor heterodimerization with erbB-3 and/or erbB-4 (Carraway et al., 1994).

After binding of EGF the intrinsic kinase domain of the EGF-R becomes activated, phosphorylating several tyrosine residues in its cytoplasmic domain (Ullrich & Schlessinger, 1990). These phosphotyrosines serve as docking sites for proteins such as Growth Factor Receptor Bound Protein 2 (Grb2) and SHC, which have SH2 domains that couple the EGF-R to the ras pathway (Batzer et al. 1994). Activated ras triggers a cascade of serine/threonine phosphorylations, mediated by kinases such as the mitogen-activated protein kinases (MAPK) (Davis, 1993), leading to phosphorylation of specific nuclear transcription factors. Several other target proteins become phosphorylated in response to EGF, including phospholipase C-γ (PLC-γ), phosphatidyl inositol 3 kinase (PI3-kinase), the non-receptor tyrosine kinase Jak 1 and members of the signal transducers and activators of transcription family of transcription factors (STAT 1, 3, and 5) (David et al., 1996).

The SH2 domain containing proteins are a diverse group of molecules important in tyrosine kinase signaling (Pawson, 1995). The SH2 domain is a non-catalytic region of ˜100 amino acids that facilitates binding to tyrosine phosphorylated GFR (GFTKR) due to a direct interaction between the SH2 domain and the phosphotyrosine-containing regions within the cytoplasmic region of the receptor (Pawson, 1995). Some SH2 domain proteins, eg. PLC-γ have intrinsic enzymatic activity and are denoted Class I. The binding of PLC-γ to GFTKR triggers phosphorylation of the enzyme leading to breakdown of polyphosphoinositides (Rhee & Choi, 1992). Class II SH2 domain containing proteins do not contain catalytic modules and are thought to function as adapter molecules, linking separate catalytic units to receptors or other signaling proteins. For example, Grb2 is a Class II member and consists of a SH2 domain flanked by two SH3 domains. The SH2 domain binds specific tyrosine phosphorylated proteins, including the EGF-R and Shc, whilst the SH3 domain binds proline-rich sequences in the ras GDP-GTP exchanger Son of Sevenless (Pawson, 1995).

Grb7 is an SH2 containing protein, and was cloned in 1992 using the Cloning of Receptor Targets approach (CORT) (Margolis et al., 1992). In this method;, the tyrosine-phosphorylated carboxyl terminus of the EGF-R was used as the probe in a murine fetal cDNA expression library. Grb7 cDNA encodes a 2.3-kb mRNA (535 aa) that is only distributed in liver, kidney, testis, ovary and breast. This limited tissue distribution suggests that Grb7 may have an important and specialized, but at present unknown, signaling function. Grb7 maps to a region on mouse chromosome 11, which also contains the tyrosine kinase receptor HER2/erbB-2 (Stein et al., 1994). Interestingly, Grb7 was found to be co-amplified and overexpressed with erbB-2 in breast cancer cell lines and samples of primary breast cancers (Stein et al., 1994). Furthermore, Grb7 binds strongly to erbB-2 via the SH2 domain creating a tight complex.

These data suggest that this basic signaling pathway involving Grb7 may be greatly amplified in certain breast cancers. Grb7 was also found to be co-expressed with the EGF-R and erbB-2 mRNA's in advanced esophageal carcinoma (Tanaka et al., 1997). Interestingly, co-expression of Grb7 occurred with EGF-R or erbB-2 in ten out of thirty-two cancers, and was significantly related to extramural tumor invasion, suggesting a possible relationship of Grb7 signaling with expression of the erbB receptors in esophageal cancer. To date, a Grb7 knockout phenotype has not been reported.

Subsequently, other members of the Grb7 family have been isolated and characterised (See FIG. 1). Grb10 was cloned in 1995, and is highly related to Grb7 (Ooi et al., 1995). Grb10 has a 6-kb mRNA which encodes at least two protein isoforms of ˜65 and 80 kD. It is expressed in heart, kidney, lung and brain. Interestingly, Grb10 maps to chromosome 11 close to the EGF-R. However, unlike Grb7, Grb10 does not bind the EGF-R protein avidly, and there is no association with erbB-2, the EGF-receptor or the PDGF-receptor (PDGF-R). Grb14, isolated in 1996, is a 58-kD protein that contains an SH2 domain at its carboxyl terminus (Daly et al., 1996). It is more widely expressed than either Grb7 or Grb10, and is present in liver, kidney, pancreas, testis, ovary, heart and skeletal muscle. Furthermore, its expression is correlated with ER expression in breast cancer cell lines. Although a glutathione-s-transferase (GST)-fusion protein containing the SH2 domain of Grb14 binds the PDGF-R, this interaction has not been demonstrated between the two proteins in vivo. All members of the Grb7 family, including F10E9.6, a putative C. elegans gene, share significant homology in specific regions of their sequence (Ooi et al., 1995). In fact, the presence of these conserved evolutionary domains suggests that Grb7 is likely to perform an important, as yet unknown, basic signaling function.

Recent evidence suggests that specific SH2 and SH3 domain-binding proteins may play a role in transferring signals from GFTKRs to intracellular RNA (Wong et al., 1992; Fugamalli et al., 1994; Hobert et al., 1994). For example, hnRNP K, a well documented ribonucleoprotein, activates downstream pathways in the cytoplasm and nucleus after binding the SH3 domain of p95^{vav} (Hobert et al., 1994). hnRNP K is the prototype member of the KR motif-containing class of RNA-Binding Proteins (RBPs). Sam68 is another recently described RBP that has been implicated in signal transduction, and RNA metabolism (Barlat et al., 1997). Sam68 is the main tyrosine-phosphorylated and Src-associated protein in mitotic cells. Interestingly, Sam68 also contains a functional KH (hnRNP K homology) RBP domain, and binds to U-rich RNA. Tyrosine phosphorylation mediates the interaction of Sam68 with multiple SH3- and SH2-containing proteins and, most remarkably, negatively regulates it RNA-binding properties. The human and murine ras-GTPase-activated protein SH3 domain-binding proteins (G3BP) have also recently been shown to bind RNA as well as to the SH3 domain of GAP (Ooi et al., 1995; Kennedy et al., 1997). This is in contrast to hnRNP K and Sam68 which both act as bifunctional proteins that bind to SH3-domains in the cytoplasm, and then translocate to the nucleus to alter RNA metabolism. G3BP is also not found in the nucleus, and is therefore likely to have different actions on RNA. It will be appreciated by those skilled in the art that these examples have established that there is a family of bifinctional adapter proteins which play a role in signaling from various SH2/SH3 containing signaling proteins as well as binding to RNA.

We have recently isolated Grb7 in a yeast three-hybrid screening approach of a human breast cancer library where we used a cis-acting AU-rich destabilizing element of EGF-R mRNA as bait. This, together with other RNA electrophoretic gel mobility shift assay (REMSA) data, suggests that Grb7 is a new member of this family of SH2/3-domain-containing signaling proteins that bind to RNA. Moreover, Grb7 represents the first member of the family for which a specific target RNA has been identified. These data imply that Grb7 is likely to play an important role in modulating EGF-R mRNA stability.

mRNA decay is now recognized as a major control point in the regulation of gene expression (Peltz et al., 1991). The recent identification of abundant examples in diverse Eukaryote biological systems in which modulation of mRNA stability directly regulates gene expression has highlighted the importance of understanding the mechanisms involved in the post transcriptional regulation of gene expression. Specific cis-acting structural RNA motifs have been recognized that can confer instability to mRNAs under appropriate conditions. For example, the discovery that an adenine plus uridine (AU)-rich sequence (AURE) from the 3′-end of unstable GM-CSF mRNA was able to reduce the half-life of β-globin mRNA from many hours to less than 30 minutes, was a landmark finding in the field (Shaw & Kamen, 1986).

Subsequently, the pentamer AUUUA was found singly, but more commonly, in multiple repeats in the 3′-UTR of a wide variety of mRNAs coding for haematopoietic growth factors (GM-CSF, M-CSF, interferons, tumor necrosis factor-α, transcriptional activator c-jun, and proto-oncogenes c-fos, c-myc, and c-myb (Greenberg & Belasco, 1993). Recent evidence indicates that the adjacent nucleotide sequence surrounding the core pentamer, specifically a nonamer, UUAUUUA^{U}/_{A}^{U}/_{A}, modifies the ability of the pentamer to mediate rapid decay (Lagnado et al., 1994; Pinol-Roma & Dreyfuss, 1992).

A family of AU-binding trans-acting factors (AUBFs) which interact with mRNAs that contain multiple AUUUA sequences and/or an AU-rich region has been characterised from several cells (Chen & Shyu, 1995). AUBFs identified to date range in size from 19-59 kD, form stable RNA-protein complexes (RPCs) and their binding activity has been augmented by agents known to stabilize specific labile mRNAs. A few AUBFs have been cloned, including one from erythroleukaemic cells (AUF1), but its functional role remains to be definitively established.

Another AUBF, termed “AUH,” unexpectedly has intrinsic enzymatic enoyl-CoA hydratase activity (Nakagawa et al., 1995). It appears to be bifunctional, although its role in the destabilization of interleukin-3 mRNA in mast cells remains to be established. In summary, Grb7 appears to be a member of a growing family of bifunctional AUBFs. Interestingly, however, the EGF-R mRNA sequence used for bait in yeast three-hybrid screening did not contain any nonamers, but instead had two extended pentamers (AUUUUA).

Given the foregoing, it will be appreciated by those skilled in the art that the molecular mechanisms that control the expression of EGR-R are still unknown. However, the Applicants have now surprisingly found that Grb7 may act as a “shuttle” from the cell membrane to EGF-R mRNA, which is most likely bound to actin microfilaments. The Applicants have also found that Grb7 contains a mRNA binding motif which binds mRNA and overexpression of Grb7 in breast cancer cells destabilizes the EGF-R mRNA. We therefore consider that EGF will regulate the association between EGF-R mRNA and Grb7, and subsequent “shuttling.” Recent data adds an intriguing twist to this hypothesis. In A431 epidermoid cancer cells that overexpress EGF-Rs, EGF regulates the association of EGF-R protein with actin microfilaments (Van Bergen en Henegouwen et al., 1992). Furthermore, recent studies indicate that EGF-R tyrosine kinase activity is up-regulated when these receptors are associated with actin microfilaments. This raises the possibility that in addition to inducing translocation of Grb7 to EGF-R mRNA associated with actin microfilaments, EGF may co-ordinately redistribute kinase active EGF-Rs to the same region.

### SUMMARY OF THE INVENTION

In its most general aspect, the invention disclosed herein provides a mRNA binding motif which, when present, effects the stability of a mRNA.

Accordingly, in a first aspect, the present invention provides a mRNA binding motif having a nucleotide sequence as shown in SEQ ID NO:1.

In a second aspect, the present invention provides a mRNA binding motif having:

a) a nucleotide sequence as shown in SEQ ID NO:1; or

b) a biologically active fragment of the sequence in a); or

c) a nucleic acid molecule which has at least 75% sequence homology to the sequence in a) or b); or

d) a nucleic acid molecule which is capable of hybridizing to any one of the sequences in a) or b) under stringent conditions.

In a third aspect, the present invention provides a shuttle adapter polypeptide or biologically-active fragment thereof, comprising an mRNA binding motif wherein the mRNA binding motif has an amino acid sequence as shown in SEQ ID NO:2. Modified and variant forms of the shuttle adapter may be produced in vitro by means of chemical or enzymatic treatment or in vivo by means of recombinant DNA technology. Such polypeptides may differ from the native shuttle adapter, for example, by virtue of one or more amino acid substitutions, deletions or insertions, or in the extent or pattern of glycosylation, but substantially retain a biological activity of the native shuttle adapter.

In a fourth aspect, the present invention provides an antisense nucleic acid that is capable of binding to a mRNA binding motif having a nucleotide sequence as shown in SEQ ID NO:1. Preferably, the antisense sequence will inhibit the activity of the mRNA binding motif in cells when transfected into them. More preferably, the inhibition will be selected from the group consisting of cell proliferation, cell differentiation and cell viability. Most preferably, the antisense sequence has a sequence as shown in SEQ ID NO:3

In an fifth aspect, the invention provides a method of screening for a ligand able to bind to and either activate or inhibit the mRNA binding motif. Such methods include but are not limited to:

a) use of antibodies to the mRNA binding motif to immunoprecipitate the mRNA binding motif and proteins bound to the mRNA binding motif;

b) screening lambda phage expression libraries for proteins that bind mRNA binding motif peptides or fragments;

c) using cDNA sequences coding for the mRNA binding motif as a “bait” sequence in the yeast three-hybrid system to screen for binding proteins;

d) using mRNA binding motif peptides and/or fragments in solid-phase affinity binding assays such as chromatography and biosensor assays to identify proteins extracted from cells and tissues that bind to mRNA binding motif peptides and fragments; and

e) using monoclonal antibodies to the mRNA binding motif and/or fragments thereof to compete for binding of the mRNA binding motif;

f) using epitope-labelled mRNA binding motif fragment to screen for binding proteins in eukaryotic cell lysates.

In further aspects, the invention provides a method for determining the presence of a nucleic acid molecule encoding the mRNA binding motif in test samples prepared from cells, tissues, or biological fluids, comprising contacting the test sample with isolated DNA comprising all or a portion of the nucleotide coding sequence for the mRNA binding motif and determining whether the isolated DNA hybridizes to a nucleic acid molecule in the test sample. DNA comprising all or a portion of the nucleotide coding sequence for the mRNA binding motif is also used in hybridization assays to identify and to isolate nucleic acids sharing substantial sequence identity to the coding sequence for the mRNA binding motif such as nucleic acids that encode allelic variants of the mRNA binding motif.

Also provided is a method for amplifying a nucleic acid molecule comprising the mRNA binding motif that is present in a test sample, comprising the use of oligonucleotides having a portion of the nucleotide sequence for the mRNA binding motif as primers in a polymerase chain reaction.

Accordingly, the present invention provides molecules capable of binding to the mRNA binding motif.

Preferably, the molecules are either ligands or antibodies, or functional fragments thereof. Where the molecule is an antibody it is preferable that the antibody is an antagonist or an agonist of the mRNA binding motif.

It is contemplated that by using the polypeptides of the invention, or an agonist or antagonist thereof, it will be possible to effect a number of interventions into cell growth and proliferation.

In a sixth aspect, the present invention provides a fragment of the mRNA binding motif capable of eliciting an antibody that co-precipitates a mRNA binding motif ligand. Preferably, the fragment has an amino acid sequence which comprises the amino acid sequence shown in SEQ ID NO:2.

In a seventh aspect, the present invention provides an antibody elicited by a mRNA binding motif fragment according to the sixth aspect of the invention. Antibodies to the mRNA binding motif are produced by immunizing an animal with the mRNA binding motif or a fragment thereof, optionally in conjunction with an immunogenic polypeptide, and thereafter recovering antibodies from the serum of the immunized animals. Alternatively, monoclonal antibodies are prepared from cells of the immunized animal in conventional fashion. Immobilized anti-mRNA binding motif antibodies are particularly useful in the detection of the mRNA binding motif in clinical samples for diagnostic purposes. Accordingly, the antibody may be polyclonal or monoclonal, but is preferably monoclonal.

In an eighth aspect, the invention provides a polypeptide that is specifically co-precipitated by an antibody of the invention from a cell expressing a protein comprising the mRNA binding motif.

### BRIEF DESCRIPTION OF THE FIGURES

FIG. 1 shows a schematic of the Grb7 family members.

FIG. 2A, FIG. 2B, FIG. 2C, FIG. 2D, FIG. 2E and FIG. 2F show regulation of EGF-R mRNA expression by EGF in MDA-468 and BT-20 human breast cancer cells, including Northern, Western blot and actinomycin D chase assays.

FIG. 3A, FIG. 3B, FIG. 3C, FIG. 3D, FIG. 3E, FIG. 3F, FIG. 3G and FIG. 3H show a schematic of EGF-R mRNA, the clones generated for transfection and RNA electrophoretic gel mobility shift assay (REMSA), as well as data from transfections and cell free mRNA decay assay.

FIG. 4A, FIG. 4B, FIG. 4C, FIG. 4D and FIG. 4E show results of multiple transfections into breast cancer cells, and assays of mRNA decay using the LightCycler.

FIG. 5A and FIG. 5B show REMSA and UV cross-linking assays with a variety of cell extracts and riboprobes.

FIG. 6A and FIG. 6B show specificty of binding for the EGF-R mRNA probe used as bait in the yeast three-hybrid screening.

FIG. 7A, FIG. 7B, FIG. 7C and FIG. 7D show REMSA using sense and antisense DNA oligomers, as well as RNA probe mutants to define the RNA binding site within the EGF-R bait.

FIG. 8A, FIG. 8B and FIG. 8C show a schematic of the yeast three-hybrid screening method, REMSA with Grb7 and other antibodies as well as a UV cross-linking Western assay using Grb7 antibodies (SenGupta et al., 1996).

FIG. 9A shows a schematic illustrating the amino acid homology between the Grb7 family members and the KH-motif. FIG. 9B and FIG. 9C show the predicted secondary structure of the Grb7 mRNA binding motif.

FIG. 10A and FIG. 10B show a schematic of Grb7 family member GST-fusion proteins, a REMSA using GST-Grb7 fusion protein with EGF-R mRNA and REMSA with unlabeled RNA competitors.

FIG. 11A, FIG. 11B, FIG. 11C, FIG. 11D, FIG. 11E and FIG. 11F show a schematic of the GST-Grb7 mutants, REMSA using the Grb7 mutants with EGF-R 2/2A riboprobe and REMSA with different EGF-R mRNA probes with each of the mutants demonstrating RNA specificity.

FIG. 12A and FIG. 12B show the sequence of the erbB-2 riboprobe used, and a REMSA showing binding of Grb7 and Grb10 to erbB-1 and erbB-2 mRNA.

FIG. 13A and FIG. 13B show REMSA binding of Grb7 and two mutants to EGF-R and erbB-2 mRNAs, together with sequence comparisons and stem-loop plots of the RNA structures.

FIG. 14A and FIG. 14B show binding by GST-Grb10 and GST-Grb14 to erbB-2 mRNA. A competition REMSA with tRNA using Grb7-M3 mutant confirmed specificity to the erbB-2 mRNA probe.

FIG. 15A and FIG. 15B show immunoprecipitation reverse transcriptase polymerase chain reaction (IP-RT-PCR) assay using Grb7 antibodies and EGF-R primers and a western blot of EGF-R levels in cells overexpressing Grb7.

FIG. 16 shows an actinomycin D chase to determine the rate of EGF-R mRNA decay in stably transfected MDA-468 cells that over-express Grb7.

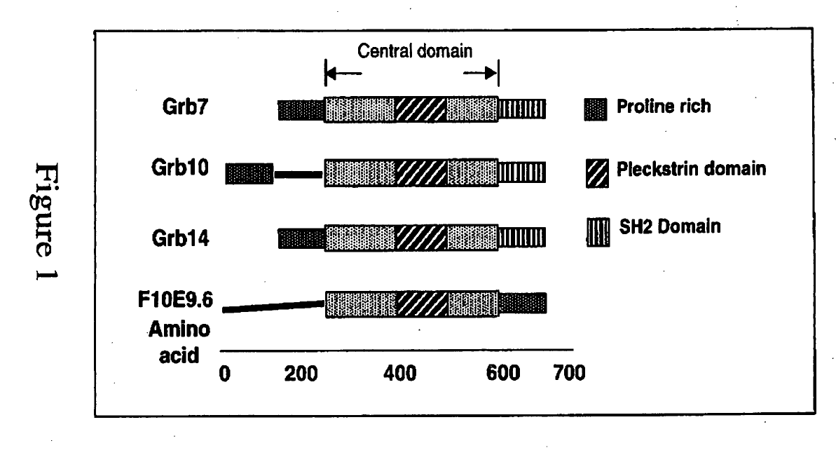

### DETAILED DESCRIPTION OF THE INVENTION

The practice of the present invention employs, unless otherwise indicated, conventional molecular biology, microbiology, and recombinant DNA techniques within the skill of the art. Such techniques are well known to the skilled worker and are explained fully in the literature. See, e.g., Maniatis, Fritsch & Sambrook, “Molecular Cloning: A Laboratory Manual” (1982); “DNA Cloning: A Practical Approach,” Volumes I and II (D. N. Glover, ed., 1985); “Oligonucleotide Synthesis” (M. J. Gait, ed., 1984); “Nucleic Acid Hybridization” (B. D. Hames & S. J. Higgins, eds., 1985); “Transcription and Translation” (B. D. Hames & S. J. Higgins, eds., 1984); “Animal Cell Culture” (R. I. Freshney, ed., 1986); “Immobilized Cells and Enzymes” (IRL Press, 1986); B. Perbal, “A Practical Guide to Molecular Cloning” (1984), and Sambrook et al., “Molecular Cloning: a Laboratory Manual” (1989).

Definitions

The description that follows makes use of a number of terms used in recombinant DNA technology. In order to provide a clear and consistent understanding of the specification and claims, including the scope given such terms, the following definitions are provided.

A “nucleic acid molecule” or “polynucleic acid molecule” refers herein to deoxyribonucleic acid and ribonucleic acid in all their forms, i.e., single and double-stranded DNA, cDNA, mRNA, and the like.

A “double-stranded DNA molecule” refers to the polymeric form of deoxyribonucleotides (adenine, guanine, thymine, or cytosine) in its normal, double-stranded helix. This term refers only to the primary and secondary structure of the molecule, and does not limit it to any particular tertiary forms. Thus, this term includes double-stranded DNA found, inter alia, in linear DNA molecules (e.g., restriction fragments), viruses, plasmids, and chromosomes. In discussing the structure of particular double-stranded DNA molecules, sequences may be described herein according to the normal convention of giving only the sequence in the 5′ to 3′ direction along the non transcribed strand of DNA (i.e., the strand having a sequence homologous to the mRNA).

A DNA sequence “corresponds” to an amino acid sequence if translation of the DNA sequence in accordance with the genetic code yields the amino acid sequence (ie., the DNA sequence “encodes” the amino acid sequence).

One DNA sequence “corresponds” to another DNA sequence if the two sequences encode the same amino acid sequence.

Two DNA sequences are “substantially similar” when at least about 85% (preferably at least about 90%, and most preferably at least about 95%) of the nucleotides match over the defined length of the DNA sequences. Sequences that are substantially similar can be identified in a Southern hybridization experiment under; for example, stringent conditions as defined for that particular system. Defining appropriate hybridization conditions is within the skill of the art. See, e.g., Maniatis et al., DNA Cloning, vols. I and II. Nucleic Acid Hybridization. However, ordinarily, “stringent conditions” for hybridization or annealing of nucleic acid molecules are those that (1) employ low ionic strength and high temperature for washing, for example, 0.015 M NaCl/0.0015 M sodium citrate/0.1% sodium dodecyl sulfate (SDS) at 50° C., or (2) employ during hybridization a denaturing agent such as formamide, for example, 50% (vol/vol) formamide with 0.1% bovine serum albumin/0.1% Ficoll/0.1% polyvinylpyrrolidone/50 mM sodium phosphate buffer at pH 6.5 with 750 mM NaCl, 75 mM sodium citrate at 42° C. Another example is use of 50% formamide, 5×SSC (0.75 M NaCl, 0.075 M sodium citrate), 50 mM sodium phosphate (pH 6.8), 0.1% sodium pyrophosphate, 5× Denhardt's solution, sonicated salmon sperm DNA (50 μg/mL), 0.1% SDS, and 10% dextran sulfate at 42° C., with washes at 42° C. in 0.2×SSC and 0.1% SDS.

A “heterologous” region or domain of a DNA construct is an identifiable segment of DNA within a larger DNA molecule that is not found in association with the larger molecule in nature. Thus, when the heterologous region encodes a mammalian gene, the gene will usually be flanked by DNA that does not flank the mammalian genomic DNA in the genome of the source organism. Another example of a heterologous region is a construct where the coding sequence itself is not found in nature (e.g., a cDNA where the genomic coding sequence contains introns, or synthetic sequences having codons different than the native gene). Allelic variations or naturally occurring mutational events do not give rise to a heterologous region of DNA as defined herein.

A coding sequence is an in-frame sequence of codons that (in view of the genetic code) correspond to or encode a protein or peptide sequence. Two coding sequences correspond to each other if the sequences or their complementary sequences encode the same amino acid sequences. A “coding sequence” in association with appropriate regulatory sequences may be transcribed and translated into a polypeptide in vivo. A polyadenylation signal and transcription termination sequence will usually be located 3′ to the coding sequence. A “promoter sequence” is a DNA regulatory region capable of binding RNA polymerase in a cell and initiating transcription, of a downstream (3′ direction) coding sequence. A coding sequence is “under the control” of the promoter sequence in a cell when RNA polymerase which binds the promoter sequence transcribes the coding sequence into mRNA which is then in turn translated into the protein encoded by the coding sequence.

A “clone” is a population of cells derived from a single cell or common ancestor by mitosis.

“Cell,” “host cell,” “cell line,” and “cell culture” are used interchangeably and all such terms should be understood to include progeny. A “cell line” is a clone of a primary cell that is capable of stable growth in vitro for many generations. Thus, the words “transformants” and “transformed cells” include the primary subject cell and cultures derived therefrom without regard for the number of times the cultures have been passaged. It should also be understood that all progeny might not be precisely identical in DNA content, due to deliberate or inadvertent mutations.

Vectors are used to introduce a foreign substance, such as DNA, RNA or protein, into an organism. Typical vectors include recombinant viruses (for DNA) and liposomes (for protein). A “DNA cloning vector” is an autonomously replicating DNA molecule, such as plasmid, phage or cosmid. Typically the DNA cloning vector comprises one or a small number of restriction endonuclease recognition sites at which such DNA sequences may be cut in a determinable fashion without loss of an essential biological function of the vector, and into which a DNA fragment may be spliced in order to bring about its replication and cloning. The cloning vector may further contain a marker suitable for use in the identification of cells transformed with the cloning vector.

An “expression vector” is similar to a DNA cloning vector but which contains regulatory sequences that will direct protein synthesis by an appropriate host cell. This usually means a promoter to bind RNA polymerase and initiate transcription of mRNA, as well as ribosome binding sites and initiation signals to direct translation of the mRNA into a polypeptide. Incorporation of a DNA sequence into an expression vector at the proper site and in correct reading frame, followed by transformation of an appropriate host cell by the vector, enables the production of mRNA corresponding to the DNA sequence, and usually of a protein encoded by the DNA sequence.

“Plasmids” are DNA molecules that are capable of replicating within a host cell, either extrachromosomally or as part of the host cell chromosome(s), and are designated by a lower case “p” preceded and/or followed by capital letters and/or numbers. The starting plasmids herein are commercially available, are publicly available on an unrestricted basis, or can be constructed from such available plasmids as disclosed herein and/or in accordance with published procedures. In certain instances, as will be apparent to the ordinarily skilled artisan, other plasmids known in the art may be used interchangeably with plasmids described herein.

“Control sequences” refers to DNA sequences necessary for the expression of an operably linked nucleotide coding sequence in a particular host cell. The control sequences that are suitable for expression in prokaryotes, for example, include origins of replication, promoters, ribosome binding sites, and transcription termination sites. The control sequences that are suitable for expression in eukaryotes, for example, include origins of replication, promoters, ribosome binding sites, polyadenylation signals, and enhancers.

An “exogenous” element is one that is foreign to the host cell, or homologous to the host cell but in a position within the host cell in which the element is ordinarily not found.

“Digestion” of DNA refers to the catalytic cleavage of DNA with an enzyme that acts only at certain locations in the DNA. Such enzymes are called restriction enzymes or restriction endonucleases, and the sites within DNA where such enzymes cleave are called restriction sites. If there are multiple restriction sites within the DNA, digestion will produce two or more linearized DNA fragments (restriction fragments). The various restriction enzymes used herein are commercially available and their reaction conditions, cofactors, and other requirements as established by the enzyme manufacturers are used. Restriction enzymes commonly are designated by abbreviations composed of a capital letter followed by other letters representing the microorganism from which each restriction enzyme originally was obtained and then a number designating the particular enzyme. In general, about 1 μg of DNA is digested with about 1-2 units of enzyme in about 20 μl of buffer solution. Appropriate buffers and substrate amounts for particular restriction enzymes are specified by the manufacturer, and/or are well known in the art.

“Recovery” or “isolation” of a given fragment of DNA from a restriction digest typically is accomplished by separating the digestion products, which are referred to as “restriction fragments,” on a polyacrylamide or agarose gel by electrophoresis, identifying the fragment of interest on the basis of its mobility relative to that of marker DNA fragments of known molecular weight, excising the portion of the gel that contains the desired fragment, and separating the DNA from the gel, for example by electroelution.

“Ligation” refers to the process of forming phosphodiester bonds between two double-stranded DNA fragments. Unless otherwise specified, ligation is accomplished using known buffers and conditions with 10 units of T4 DNA ligase per 0.5 μg of approximately equimolar amounts of the DNA fragments to be ligated.

“Oligonucleotides” are short-length, single- or double-stranded polydeoxynucleotides that are chemically synthesized by known methods (involving, for example, triester, phosphoramidite, or phosphonate chemistry), such as described by Engels, et al. (1989). They are then purified, for example, by polyacrylamide gel electrophoresis.

“Polymerase chain reaction,” or “PCR™,” as used herein generally refers to a method for amplification of a desired nucleotide sequence in vitro, as described in U.S. Pat. No. 4,683,195. In general, the PCR™ method involves repeated cycles of primer extension synthesis, using two oligonucleotide primers capable of hybridizing preferentially to a template nucleic acid. Typically, the primers used in the PCR™ method will be complementary to nucleotide sequences within the template at both ends of or flanking the nucleotide sequence to be amplified, although primers complementary to the nucleotide sequence to be amplified also may be used. Wang et al., 1990; Ochman, et al., 1990; Triglia, et al., 1988.

“PCR™ cloning” refers to the use of the PCR™ method to amplify a specific desired nucleotide sequence that is present amongst the nucleic acids from a suitable cell or tissue source, including total genomic DNA and cDNA transcribed from total cellular RNA. Frohman et al., 1988; Saiki et al., 1988; Mullis et al., 1987.

Nucleotide sequence variants of the mRNA binding motif may be prepared by a variety of methods known in the art. These methods include, but are not limited to, isolation from a natural source (in the case of naturally occurring nucleotide sequence variants of the mRNA binding motif) or preparation by site-directed (or oligonucleotide-mediated) mutagenesis, PCR™ mutagenesis, and cassette mutagenesis of an earlier prepared DNA encoding a variant or a non-variant form of the mRNA binding motif.

Site-directed mutagenesis is a preferred method for preparing substitution, deletion, and insertion variants of the mRNA binding motif DNA. This technique is well known in the art (Zoller et al., 1983; Zoller et al., 1987; Carter, 1987; Horwitz et al., 1990), and has been used, for example, to produce amino acid sequence variants of trypsin and T4 lysozyme, which variants have certain desired functional properties. Perry et al., 1984; Craik et al., 1985.

Briefly, in carrying out site-directed mutagenesis of the mRNA binding motif DNA, the mRNA binding motif DNA is altered by first hybridizing an oligonucleotide encoding the desired mutation to a single strand of such the mRNA binding motif DNA. After hybridization, a DNA polymerase is used to synthesize an entire second strand, using the hybridized oligonucleotide as a primer, and using the single strand of the mRNA binding motif as a template. Thus, the oligonucleotide encoding the desired mutation is incorporated in the resulting double-stranded DNA.

Oligonucleotides for use as hybridization probes or primers may be prepared by any suitable method, such as by purification of a naturally occurring DNA or by in vitro synthesis. For example, oligonucleotides are readily synthesized using various techniques in organic chemistry, such as described by Narang et al., 1979; Brown et al., 1979; Caruther et al., 1985. The general approach to selecting a suitable hybridization probe or primer is well known. Keller et al., 1989. Typically, the hybridization probe or primer will contain 10-25 or more nucleotides, and will include at least 5 nucleotides on either side of the sequence encoding the desired mutation so as to ensure that the oligonucleotide will hybridize preferentially to the single-stranded DNA template molecule.

Multiple mutations are introduced into the mRNA binding motif DNA to produce nucleotide sequence variants of the mRNA binding motif comprising several or a combination of insertions, deletions, or substitutions of nucleotides as compared to the nucleotide sequences set forth in SEQ ID NO.:1. If the sites to be mutated are located close together, the mutations may be introduced simultaneously using a single oligonucleotide that encodes all of the desired mutations. If, however, the sites to be mutated are located some distance from each other (separated by more than about ten nucleotides), it is more difficult to generate a single oligonucleotide that encodes all of the desired changes. Instead, one of two alternative methods may be employed.

In the first method, a separate oligonucleotide is generated for each desired mutation. The oligonucleotides are then annealed to the single-stranded template DNA simultaneously, and the second strand of DNA that is synthesized from the template will encode all of the desired nucleotide substitutions.

The alternative method involves two or more rounds of mutagenesis to produce the desired mutant. The first round is as described for introducing a single mutation: a single strand of a previously prepared DNA is used as a template, an oligonucleotide encoding the first desired mutation is annealed to this template, and a heteroduplex DNA molecule is then generated. The second round of mutagenesis utilizes the mutated DNA produced in the first round of mutagenesis as the template. Thus, this template already contains one or more mutations. The oligonucleotide encoding the additional desired nucleotide substitution(s) is then annealed to this template, and the resulting strand of DNA now encodes mutations from both the first and second rounds of mutagenesis. This resultant DNA can be used as a template in a third round of mutagenesis, and so on.

PCR™ mutagenesis is also suitable for making nucleotide sequence variants of the mRNA binding motif. Higuchi, 1990; Vallette et al., 1989. Briefly, when small amounts of template DNA are used as starting material in a PCR™, primers that differ slightly in sequence from the corresponding region in a template DNA can be used to generate relatively large quantities of a specific DNA fragment that differs from the template sequence only at the positions where the primers differ from the template. For introduction of a mutation into a plasmid DNA, for example, one of the primers is designed to overlap the position of the mutation and to contain the mutation; the sequence of the other primer must be identical to a nucleotide sequence within the opposite strand of the plasmid DNA, but this sequence can be located anywhere along the plasmid DNA. It is preferred, however, that the sequence of the second primer is located within 200 nucleotides from that of the first, such that in the end the entire amplified region of DNA bounded by the primers can be easily sequenced. PCR™ amplification using a primer pair like the one just described results in a population of DNA fragments that differ at the position of the mutation specified by the primer, and possibly at other positions, as template copying is somewhat error-prone. Wagner et al., 1991.

If the ratio of template to product amplified DNA is extremely low, the majority of product DNA fragments incorporate the desired mutation(s). This product DNA is used to replace the corresponding region in the plasmid that served as PCR™ template using standard recombinant DNA methods. Mutations at separate positions can be introduced simultaneously by either using a mutant second primer, or performing a second PCR™ with different mutant primers and ligating the two resulting PCR™ fragments simultaneously to the plasmid fragment in a three (or more)-part ligation.

Another method for preparing variants, cassette mutagenesis, is based on the technique described by Wells et al., 1985. The starting material is a plasmid (or other vector) comprising the mRNA binding motif DNA to be mutated. The codon(s) in the mRNA binding motif DNA to be mutated are identified. There must be a unique restriction endonuclease site on each side of the identified mutation site(s). If no such restriction sites exist, they may be generated using the above-described oligonucleotide-mediated mutagenesis method to introduce them at appropriate locations in the mRNA binding motif DNA. The plasmid DNA is cut at these sites to linearize it. A double-stranded oligonucleotide encoding the sequence of the DNA between the restriction sites but containing the desired mutation(s) is synthesized using standard procedures, wherein the two strands of the oligonucleotide are synthesized separately and then hybridized together using standard techniques. This double-stranded oligonucleotide is referred to as the cassette. This cassette is designed to have 5′ and 3′ ends that are compatible with the ends of the linearized plasmid, such that it can be directly ligated to the plasmid. This plasmid now contains the mutated mRNA binding motif.

As used herein, the terms “transformation” and “transfection” refer to the process of introducing a desired nucleic acid, such a plasmid or an expression vector, into a host cell. Various methods of transformation and transfection are available, depending on the nature of the host cell. In the case of E. coli cells, the most common methods involve treating the cells with aqueous solutions of calcium chloride and other salts. In the case of mammalian cells, the most common methods are transfection mediated by either calcium phosphate or DEAE-dextran, or electroporation. Sambrook et al., 1989. Following transformation or transfection, the desired nucleic acid may integrate into the host cell genome, or may exist as an extrachromosomal element.

The mRNA binding motif may be used as an immunogen to generate anti-mRNA binding motif antibodies. Such antibodies, which specifically bind to the mRNA binding motif, are useful as standards in assays for the mRNA binding motif. Ordinarily, the anti-mRNA binding motif antibody will bind the mRNA binding motif with an affinity of at least about 10⁶ L/mole, and preferably at least about 10⁷ L/mole.

Polyclonal antibodies directed toward the mRNA binding motif generally are raised in animals by multiple subcutaneous or intraperitoneal injections of the mRNA binding motif peptide and an adjuvant. It may be useful to conjugate the mRNA binding motif or a peptide fragment thereof to a carrier protein that is immunogenic in the species to be immunized, such as keyhole limpet hemocyanin, serum albumin, bovine thyroglobulin, or soybean trypsin inhibitor, using a bifunctional or derivatizing agent, for example, maleimidobenzoyl sulfosuccinimide ester (conjugation through cysteine residues), N-hydroxysuccinimide (conjugation through lysine residues), glutaraldehyde, succinic anhydride, SOCl₂, or R¹N═C═NR, where R and R¹ are different alkyl groups.

Animals are immunized with the mRNA binding motif-carrier protein conjugates combining 1 mg or 1 μg of conjugate (for rabbits or mice, respectively) with 3 volumes of Freund's complete adjuvant and injecting the solution intradermally at multiple sites. One month later the animals are boosted with ⅕th to 1/10th the original amount of conjugate in Freund's complete adjuvant by subcutaneous injection at multiple sites. 7 to 14 days later animals are bled and the serum is assayed for anti-mRNA binding motif antibody titer. Animals are boosted until the antibody titer plateaus. Preferably, the animal is boosted by injection with a conjugate of the same the mRNA binding motif with a different carrier protein and/or through a different cross-linking agent. Conjugates of the mRNA binding motif and a suitable carrier protein also can be made in recombinant cell culture as fusion proteins. Also, aggregating agents such as alum are used to enhance the immune response.

Monoclonal antibodies directed toward the mRNA binding motif are produced using any method that provides for the production of antibody molecules by continuous cell lines in culture. The modifier “monoclonal” indicates the character of the antibody as being obtained from a substantially homogeneous population of antibodies, and is not to be construed as requiring production of the antibody by any particular method. Examples of suitable methods for preparing monoclonal antibodies include the original hybridoma method of Kohler et al., 1975, and the human B-cell hybridoma method, Kozbor, 1984; Brodeur et al., 1987.

The monoclonal antibodies of the invention specifically include “chimeric” antibodies (immunoglobulins) in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity (U.S. Pat. No. 4,816,567; Morrison et al., 1984).

In a preferred embodiment, the chimeric anti-mRNA binding motif antibody is a “humanized” antibody. Methods for humanizing non-human antibodies are well known in the art. Generally, a humanized antibody has one or more amino acid residues introduced into it from a source that is non-human. These non-human amino acid residues are often referred to as “import” residues, which are typically taken from an “import” variable domain.

Humanization can be performed following methods known in the art (Jones et al., 1986; Riechmann et al., 1988; Verhoeyen et al., 1988), by substituting rodent complementarity-determining regions (CDRs) for the corresponding regions of a human antibody. Alternatively, it is now possible to produce transgenic animals (e.g., mice) that are capable, upon immunization, of producing a full repertoire of human antibodies in the absence of endogenous immunoglobulin production. For example, it has been described that the homozygous deletion of the antibody heavy-chain joining region (J_{H}) gene in chimeric and germ-line mutant mice results in complete inhibition of endogenous antibody production. Transfer of the human germ-line immunoglobulin gene array in such germ-line mutant mice will result in the production of human antibodies upon antigen challenge. See, for example, Jakobovits et al., 1993; Jakobovits et al., 1993; Bruggermann et al., 1993. Human antibodies can also be produced in phage-display libraries (Hoogenboom et al., 1991; Marks et al., 1991).

For diagnostic applications, anti-mRNA binding motif antibodies typically will be labeled with a detectable moiety. The detectable moiety can be any one which is capable of producing, either directly or indirectly, a detectable signal. For example, the detectable moiety may be a radioisotope, such as ³H, ¹⁴C, ³²P, ³⁵S, or ¹²⁵I, a fluorescent or chemiluminescent compound, such as fluorescein isothiocyanate, rhodamine, or luciferin; radioactive isotopic labels, such as, e.g., ¹²⁵I, ³²P, ¹⁴C, or ³H, or an enzyme, such as alkaline phosphatase, beta-galactosidase or horseradish peroxidase.

Any method known in the art for separately conjugating the antibody to the detectable moiety may be employed, including those methods described by David et al., 1974; Pain et al., 1981; and Bayer et al., 1990.

The anti-mRNA binding motif antibodies may be employed in any known assay method, such as competitive binding assays, direct and indirect sandwich assays, and immunoprecipitation assays. Zola, 1987.

Competitive binding assays rely on the ability of a labeled standard (e.g., the mRNA binding motif or an immunologically reactive portion thereof) to compete with the test sample analyte (the mRNA binding motif) for binding with a limited amount of antibody. The amount of the mRNA binding motif in the test sample is inversely proportional to the amount of standard that becomes bound to the antibodies. To facilitate determining the amount of standard that becomes bound, the antibodies generally are insolubilized before or after the competition, so that the standard and analyte that are bound to the antibodies may conveniently be separated from the standard and analyte which remain unbound.

Sandwich assays involve the use of two antibodies, each capable of binding to a different immunogenic portion, or epitope, of the protein to be detected. In a sandwich assay, the test sample analyte is bound by a first antibody which is immobilized on a solid support, and thereafter a second antibody binds to the analyte, thus forming an insoluble three part complex. U.S. Pat. No. 4,376,110. The second antibody may itself be labeled with a detectable moiety (direct sandwich assays) or may be-measured using an anti-immunoglobulin antibody that is labeled with a detectable moiety (indirect sandwich assay). For example, one type of sandwich assay is an ELISA assay, in which case the detectable moiety is an enzyme.

The anti-mRNA binding motif antibodies of the invention also are useful for in vivo imaging, wherein an antibody labeled with a detectable moiety is administered to a host, preferably into the bloodstream, and the presence and location of the labeled antibody in the host is assayed. The antibody may be labeled with any moiety that is detectable in a host, whether by nuclear magnetic resonance, radiology, or other detection means known in the art.

Neutralizing anti-mRNA binding motif antibodies are useful as antagonists of the mRNA binding motif. The term “neutralizing anti-mRNA binding motif antibody” as used herein refers to an antibody that is capable of specifically binding to the mRNA binding motif, and which is capable of substantially inhibiting or eliminating the functional activity of the mRNA binding motif in vivo or in vitro. Typically a neutralizing antibody will inhibit the functional activity of the mRNA binding motif at least about 50%, and preferably greater than 80%, as determined, for example, by an in vitro receptor binding assay.

Shuttle adapter refers to the ability of Grb7 to shuttle between the cell membrane and the cytoplasm. Grb7 binds to growth factor receptors (EGF-R and erbB-2) at the cell membrane. We propose that it then moves away from the cell membrane “shuttling” into the cytoplasm where it then binds mRNA, which may be localized to actin microfilaments. The Grb7-mRNA complex is then transported to the ribosomes. In each case, Grb7 serves different functions at either the cell membrane or cytoplasm. We propose that Grb7 binds protein (via a 3′ end of the SH2 domain interaction) at or near the cell membrane and to mRNA in the cytoplasm via the mRNA-binding domain.

The invention will now be further described by way of reference only to the following non-limiting examples. It should be understood, however, that the examples following are illustrative only, and should not be taken in any way as a restriction on the generality of the invention described above. Amino acid sequences referred to herein are given in standard single letter code.

### EXAMPLE 1

Ligand-Induced Regulation of EGF-R mRNA Stability in Breast, Prostate and Epidermoid Cancer Cells

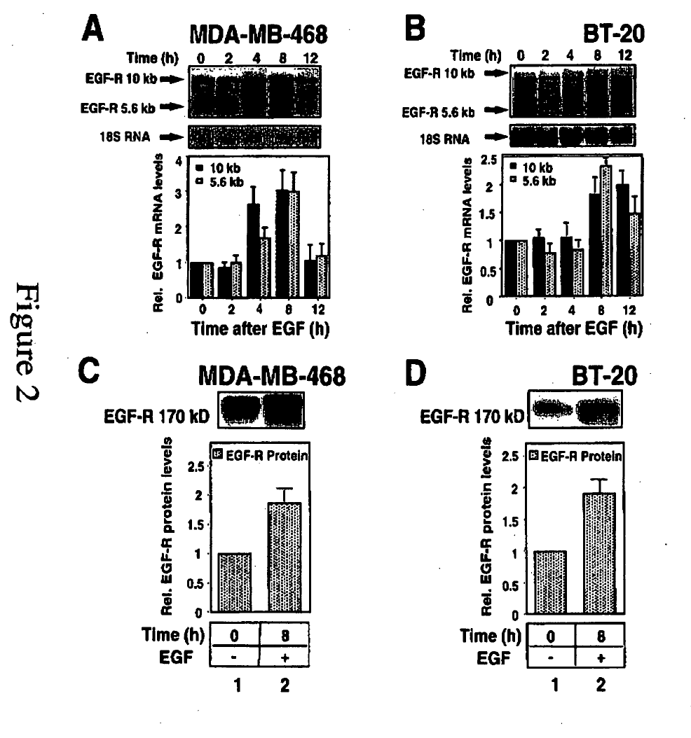

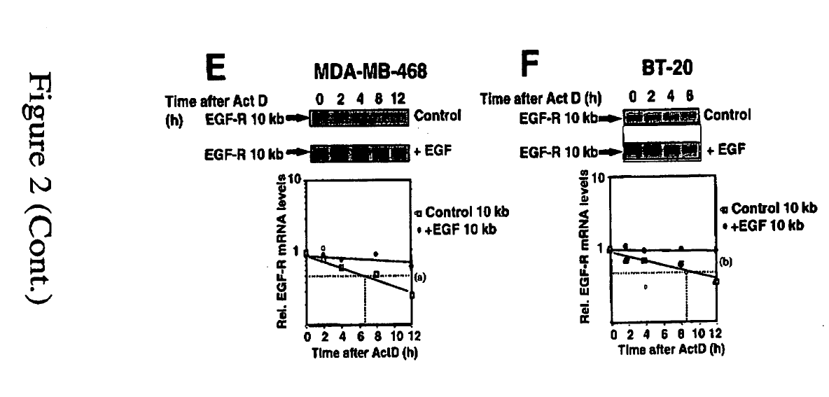

We have recently shown that mRNA turnover plays a major role in the regulation of EGF-R gene expression in a variety of cancer cell lines. EGF upregulates EGF-R mRNA and protein in breast cancer cells (Balmer et al., 2000) (FIG. 2A, FIG. 2B, FIG. 2C and FIG. 2D). Furthermore, EGF stabilizes EGF-R mRNA in the breast cancer cell lines MDA-468, BT-20 (Balmer et al., 2000), prostate cancer cell lines LNCaP and DU-145 (Seth et al. 1999), and epidermoid cancer cell line KB (McCulloch et al. 1998). As can be seen in FIG. 2A, FIG. 2B, FIG. 2C, FIG. 2D, FIG. 2E and FIG. 2F, in the MDA-468 and BT-20 breast cancer cell lines, EGF stabilized EGF-R mRNA stability and induced a concomitant increase in EGF-R protein level.

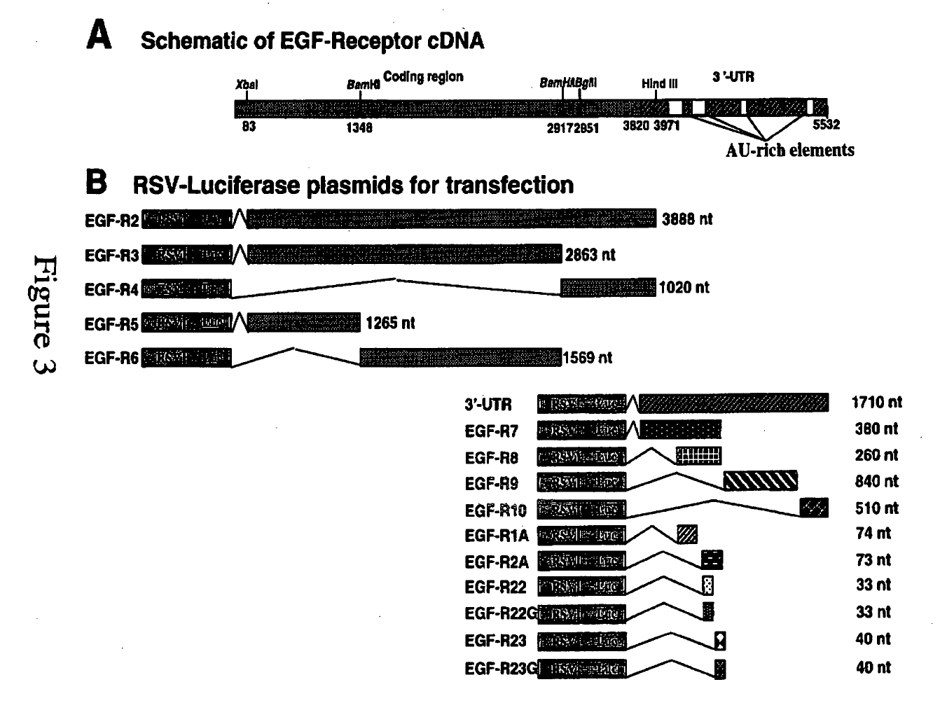

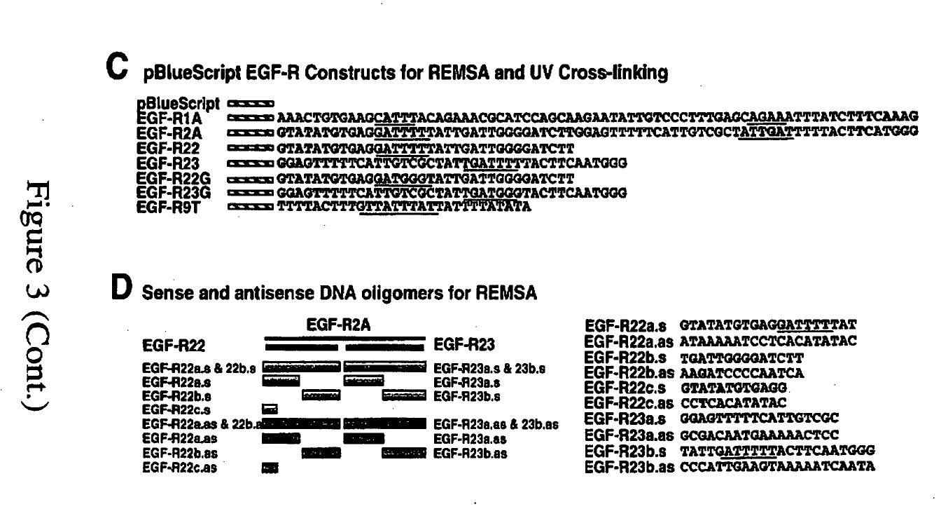

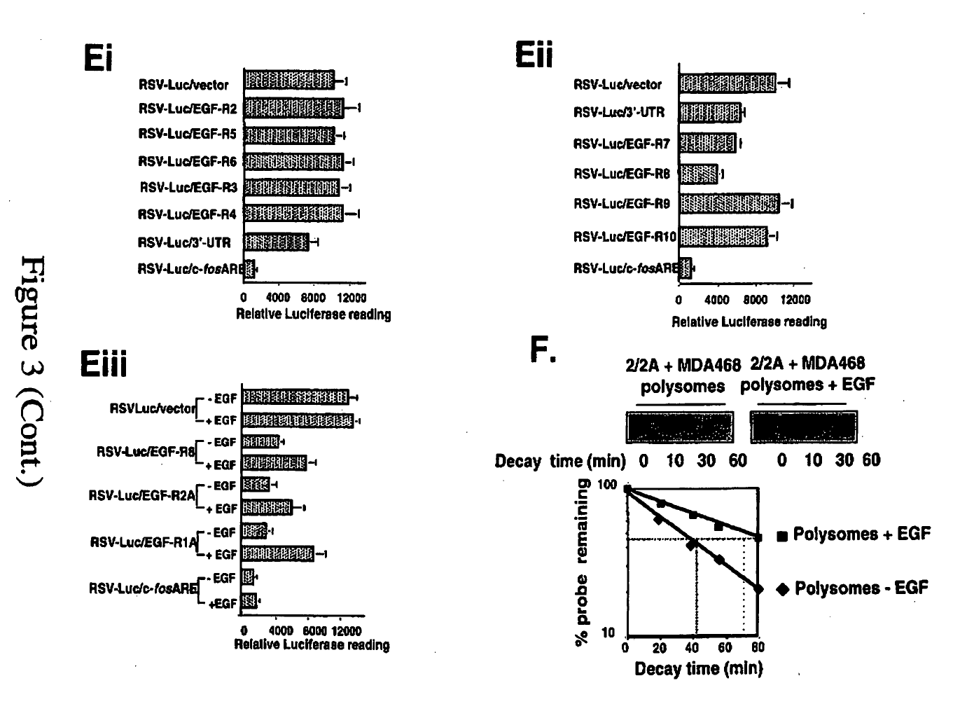

Using transfection studies in MDA-468 breast cancer cells, we identified a novel 259 bp AURE (SEQ ID NO:4; Table 1) within the 3′-UTR of EGF-R mRNA that reduced the basal activity of a heterologous luciferase reporter construct (RSV-Luc). This 259-bp AURE contains two ˜80-bp regions (¹/_{1A} & ²/_{2A}), each of which has two AU-rich sequences, the most 5′ of which contains 2 AUUUA pentamers, the other, 2 extended pentamers AUUUUUA (See FIG. 3A, FIG. 3B, FIG. 3C and FIG. 3D).

TABLE 1

CCGACTAGCCAGGAAGTACTTCCACCTCGGGCACATTTTGGGAAGTTGCATTCCTTTGTCTTCAAACTGT
GAAGCATTTACAGAAACGCATCCAGCAAGAATATTGTCCCTTTGAGCAGAAATTTATCTTTCAAAGAGGT
ATATTTGAAAAAAAAAAAAAAAGTATATGTGAGGATTTTTATTGATTGGGGATCTTGGAGTTTTTCATTG
TCGCTATTGATTTTTACTTCAATGGGCTCTTCCAACAAGGAAGAAGCTT-3′

Nucleotide Sequence of EGF-R mRNA 3′UTR AU-rich region (SEQ ID NO: 4). From nt 3954 to 4212 (259 nt) of human EGF-R. AU-pentamers and extended pentamers are underlined. The sequence of 1/1A (nt 4016-4089, 74 nt) and 2/2A (nt 4116-4189, 74 nt) are bolded.

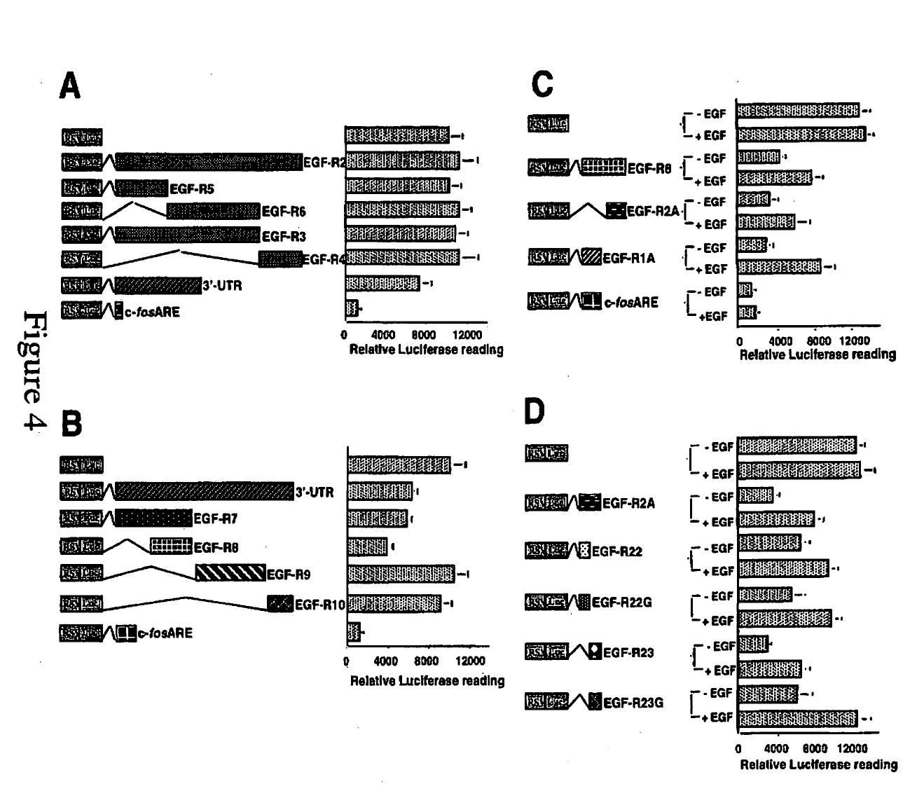

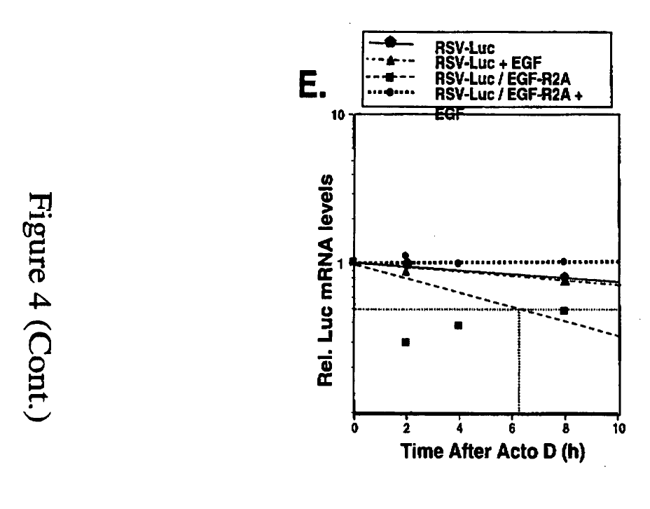

In MDA-468 breast cancer cells, each of these 80-bp regions also decreases basal activity of the heterologous reporter which is positively regulated by EGF (See FIG. 3E, FIG. 4C and FIG. 4D). The full-length ²/_{2A} probe is 73 nucleotides.

To verify that the luciferase (Luc) reporter assay was representative of changes in Luc mRNA turnover, we performed a parallel set of transfections to determine the Luc mRNA decay rate using Actinomycin D chase, RT-PCR and the LightCycler. We found that the half-life of RSV-Luc mRNA was long (>14 h) (See FIG. 4E). However, the half-life of RSV-Luc/EGF-2/2A mRNA was significantly shortened to ˜6 h and EGF increased the half-life to well over 14 h, whilst having no effect on the vector alone (See FIG. 4E). Similar EGF-responsive data were obtained for RSV-Luc/EGF-R1A in this system (˜2-fold change with addition of EGF) (data not shown). Taken together, these data verify the reporter assays above, and confirm that the EGF-R1/1A and EGF-R2/2A regions act as cis elements within the EGF-R mRNA.

Similar results were obtained in a cell-free mRNA decay assay using EGF-R ²/_{2A} as a probe, in which EGF was shown to stabilize the ²/_{2A} RNA (See FIG. 3F).

### EXAMPLE 2

ER Negative Breast and Other Cancer Cells Contain RBPs Specific for Human EGF-R mRNA

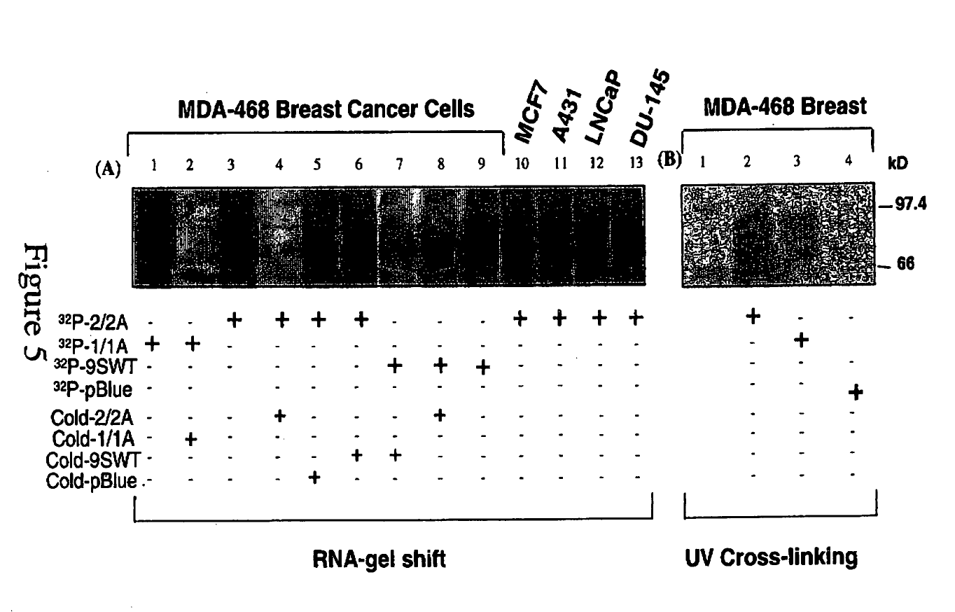

REMSA studies, using a ³²P-labeled riboprobe containing either the ¹/_{1A} or ²/_{2A} sequence of the cis-acting element, have shown that multiple cancer cell lines contain cytoplasmic AUBFs that bind specifically to these regions (See FIG. 5A). These include breast (MDA-468, BT20, MCF-7), epidermoid (A431, KB), and prostate (LNCaP, DU-145) cancer cells, indicating that the proteins are widely distributed in many tissues. Further, the binding activity of these AUBFs is regulated by EGF. UV cross-linking assays (UCAs) in MDA-468 breast cancer cells revealed that the RNA-protein complexes (RPCs) identified in REMSAs contained several different proteins (See FIG. 5B, ˜60, 70-75 and 85-90 kD). Interestingly, ¹/_{1A} bound the two larger RBPs with similar affinity to ²/_{2A}, but displayed little binding of the smaller proteins ˜60-75 kD (See FIG. 5B). It was also noted that a standard nonamer consensus probe formed a different RPC to that of ²/_{2A} (See FIG. 5A, lane 9), which was competed by cold excess nonamer probe (lane 7), but not by excess cold ²/_{2A} probe (lane 8). This suggested that the RBPs binding to ²/_{2A} are not previously identified nonamer binding AUBFs.

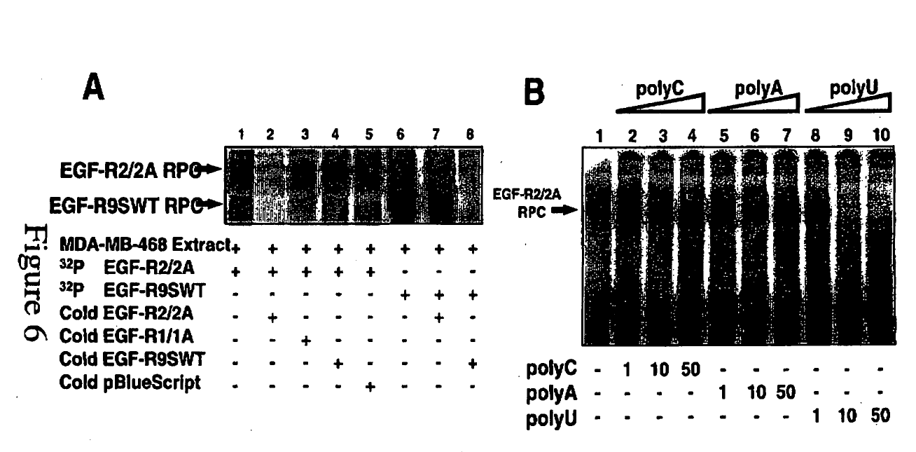

To determine the specificity of binding for the EGF-R probes, REMSA was performed with the addition of excess unlabeled competitor RNA. With the ²/_{2A} probe, the RPC was specifically competed out by excess unlabeled ²/_{2A} probe, but not by other unrelated RNA competitors (See FIG. 6A). In addition, the ²/_{2A} RPC detected in MDA-468 cells was specifically competed out with excess unlabeled polyU, but not polyA or polyC homoploymers, suggesting that the binding site was likely to be the U-rich sequence of the ²/_{2A} probe (See FIG. 6B).

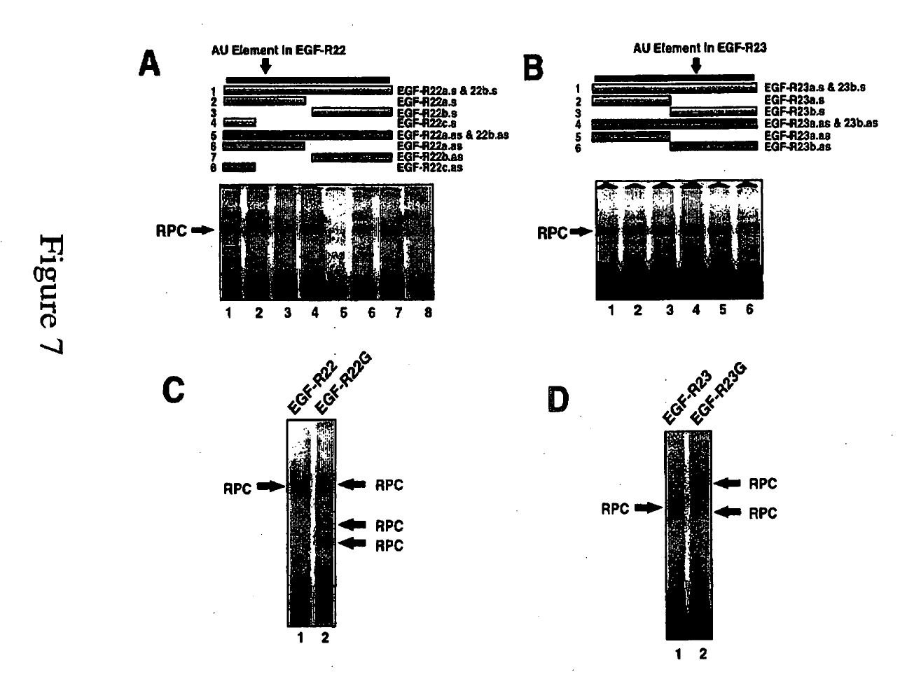

To further define the protein binding site within the ²/_{2A} probe, we generated two shorter probes, EGF-R22 and EGF-R23, corresponding to the 5′ and 3′ ends, respectively, of ²/_{2A} (see FIG. 3C and FIG. 3D). Additional probes were generated that contained mutations within the AUUUUUA sequence to AUGGGUA for each smaller probe (EGF-R22G and EGF-R23G) (See FIG. 3C). REMSA showed that the majority of binding to EGF-R22 resided in the 5′ 12 nucleotides, which did not involve the AU rich sequence (See FIGS. 7A&C). However, and in contrast, the AU-rich sequence in EGF-R23 was a significant contributor to overall binding in REMSA (See FIG. 7B and FIG. 7D).

### EXAMPLE 3

Cloning of GRB7 with the Yeast Three-Hybrid System

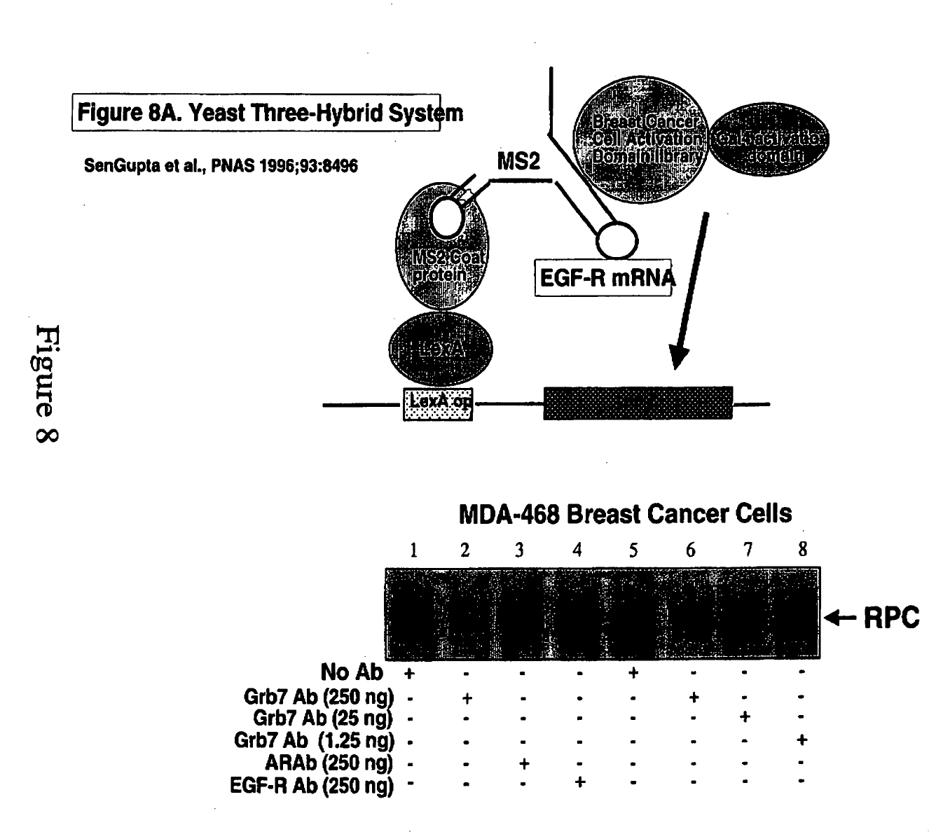

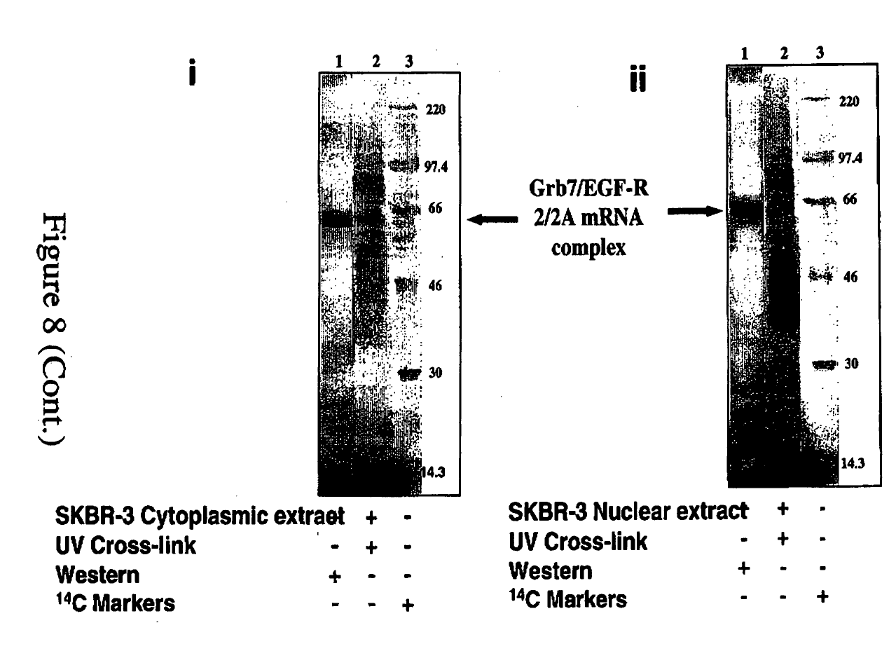

The recently described yeast three-hybrid system (SenGupta et al., 1996) facilitates detection and analysis of RNA-protein interactions in vivo. It relies on the physical, not biological, properties of the RNA and protein molecules. In principle, it shares many of the strengths of the highly successful yeast two-hybrid system. In the three-hybrid system (See FIG. 8A), a hybrid RNA molecule functions as the bridge between two hybrid proteins. Hybrid protein 1 contains RNA-binding domain 1 fused to a DNA-binding domain, while Hybrid protein 2 contains a different RNA-binding domain, 2, fused to a transcription activation domain. The hybrid RNA contains recognition sites for the two RNA-binding domains. The interaction of this RNA with the two hybrid proteins is required for transcription of the reporter gene. We screened a human breast cancer activation domain cDNA library using the EGF-R ²/_{2A} sequence as bait in the three-hybrid system.

The cDNA library was provided by Dr Jennifer Byrne. It was generated from a human primary infiltrating ductal breast carcinoma. It was constructed in the Hybri-Zap™ vector (Stratgene) using pAD-GAL4 to express GAL4 (761-881) activation domain fusion proteins from the ADH1 promoter. Over 90% of pAD-GAL4 plasmids contain inserts, the average insert size being 1.1 kb.

One clone encoded the SH2 domain-containing signaling molecule Grb7. Grb7 encodes a protein of ˜58 kD. This corresponds well with our identification of a band at ˜60 kD (lowest band) in UCA with the ²/_{2A} probe (Grb7 ˜58 kD plus a component of the RNA probe ˜2-5 kD). To determine whether Grb7 was involved in the EGF-R RPC, REMSA was performed using a labelled ²/_{2A} probe, cell cytoplasmic extract from either MDA-468 of SKBR-3 breast cancer cells and one of several antibodies (Ab), including a rabbit polyclonal Grb7 antibody (Ab) (Transduction Laboratories, G22830). As can be seen in FIG. 8B, addition of Grb7 Ab significantly reduced RPC formation (lane 2), whilst no effect was seen with either EGF-R or androgen receptor Abs (lanes 3 & 4). Furthermore, an Ab titration REMSA showed that as the Grb7 Ab concentration was decreased the intensity of the RPC complex increased (See FIG. 8B, lanes 6-8), providing strong evidence for a direct effect of Grb7 on the formation of the EGF-R RPC. Similar results were obtained in the SKBR-3 cell cytoplasmic cell extracts. We also examined cell extracts for binding of Grb7 to EGF-R 2/2A mRNA in UV cross-link/Western assays. As can be seen in FIG. 8C, an RNA-protein complex was observed in both cytoplasmic and nuclear extracts of SKBR-3 breast cancer cells at ˜58-60 kD (FIG. 8C, lane 2 in each Panel). When the UV cross-linked RNA-protein complexes were transferred to a membrane and the membrane probed with a Grb7 antibody, a strong band at the same Mr (˜58-60 kD) was identified (FIG. 8C, lane 1 in each Panel). These results provided further evidence for the binding of Grb7 to EGF-R mRNA.

Taken together, these data suggest that Grb7 is a novel EGF-R RBP. This discovery is remarkable given that Grb7 was originally cloned because of its ability to bind to the EGF-R protein.

### EXAMPLE 4

GRB7 Contains an RNA-Binding Protein Domain

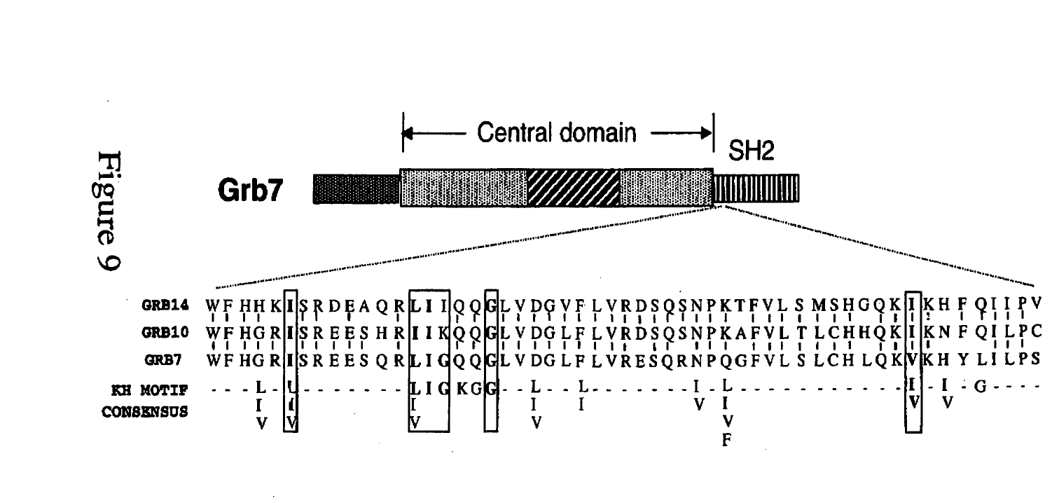

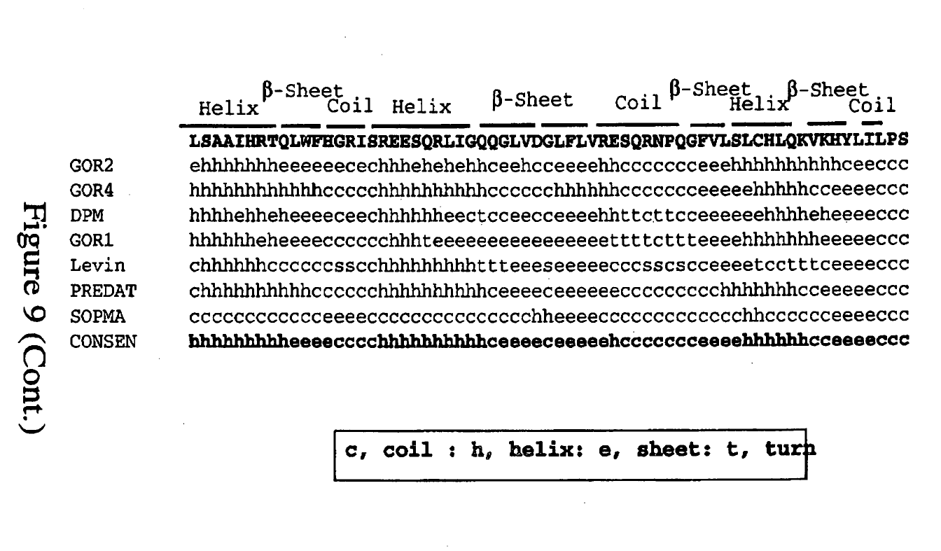

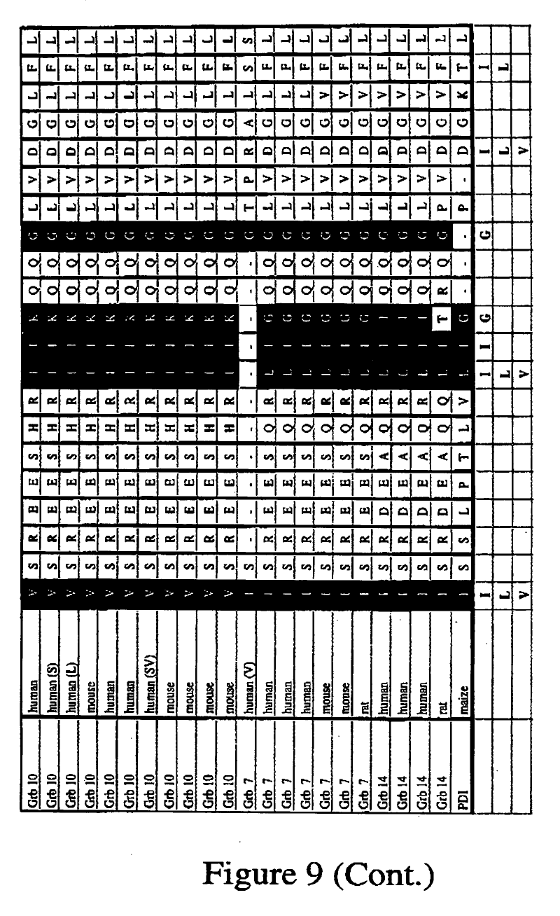

Although the Grb7 protein sequence does not contain any previously recognised RNA-binding domain, a region near the 5′ end of the SH2 domain shares some features of a K-homology (KH) domain (FIG. 9A). The KH motif is the most recent addition to the collection of motifs in RNA-binding proteins, and was first identified in the human hnRNP K protein. The widespread presence of KH motifs in diverse organisms suggest that it is an ancient protein structure with important cellular functions. Protein′ family members include ribosomal S3 proteins from divergent organisms and several human RBPs, including Sam68. All KH motif proteins of known function are associated with RNA, and many bind RNA in vitro (predominantly homo-polymers of U). Recent observations indicate that the KH motif is essential for RNA binding and most likely binds RNA directly via a distinct RNA-binding pocket.

The three dimensional (3D) structure of a KH domain in the vigilin-6 protein was recently determined by NMR spectroscopy (Musco et al., 1996). The KH domain consists of a βααββα fold, although there is much variation in amino acid use within these regions. Several important residues include an L and I (boxed in FIG. 9A) within the 5′ α-helix which form the putative RNA-binding pocket. The GxxG immediately 3′ of above residues is also critical as it maintains the structure at the end of the RNA-binding pocket. As can be seen in FIG. 9A, the mRNA-binding domain of Grb7 shares some of the KH domain conserved amino acids (LI and GxxG). However, there is major divergence of the Grb7 family of proteins either side of the boxed residues at the 5′ end of the sequence, consistent with some homology to, but marked difference from, the consensus KH-domain. This is reflected in a different overall predicted secondary structure for the putative Grb7 mRNA-binding domain.

Analysis of the mRNA binding motif of Grb7 by several different secondary structure prediction programs, including SOPMA and PHD alignment, has generated a consensus structure with a βαββα fold (FIG. 9B). There is marked divergence of the sequence 3′ of the 5′ central a-helix from a consensus KH-domain, consistent with the difference in predicted folding pattern. The marked difference in sequence and folding of the critical second and third a helices between the Grb7 mRNA-binding domain and the KH domain suggests completely different RNA-binding specificities with each domain. The putative RNA-binding core of Grb7 is thus very different from the consensus KR-binding domain.

To determine the sequence homology across all of the known sequences available for the Grb7 family members, we performed a sequence alignment analysis. As can be seen in FIG. 9C, there is significant sequence divergence within the family members at the glycine (G) residue within the LIG motif. Otherwise there is strict conservation across the KR-like domain. This suggests that each family member will bind RNA, potentially with different specificity provided by the variation within the LIG motif.

The mRNA binding motif of Grb7 lies at the 5′ end of the SH2 domain sequence (See FIG. 9A). It is unclear at present whether this 5′ SH2 domain sequence, which encompasses the mRNA binding motif, is required for binding to GFTKRs as mutational studies to date have only involved amino acids C-terminal of this region. It is possible, therefore, that the mRNA binding motif has been positioned within the SH2 domain to ensure that Grb7 can bind to either an erbB receptor at the cell surface or a specific RNA in the cytoplasm but not both at the same time.

We propose that Grb7 acts as a bifinctional protein that binds to GFRTKs at the cell membrane and then after ligand stimulation shuttles to EGF-R mRNA in the cytoplasm resulting in coordinate control of signaling and expression (mRNA turnover). cl EXAMPLE 5

### Binding of GST-GRB7 Fusion Proteins to EGF-R mRNA

Several GST-Grb7 fusion proteins were generated to examine the binding capacity to EGF-R mRNA. These included proteins that encompassed the only the SH-2 domain of Grb7, Grb10 and Grb14.

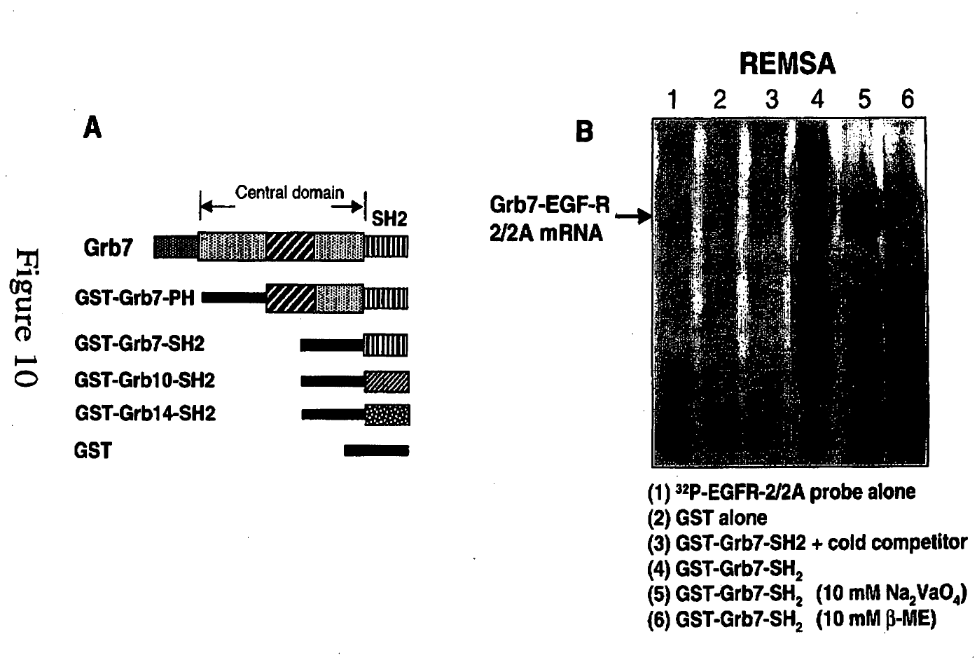

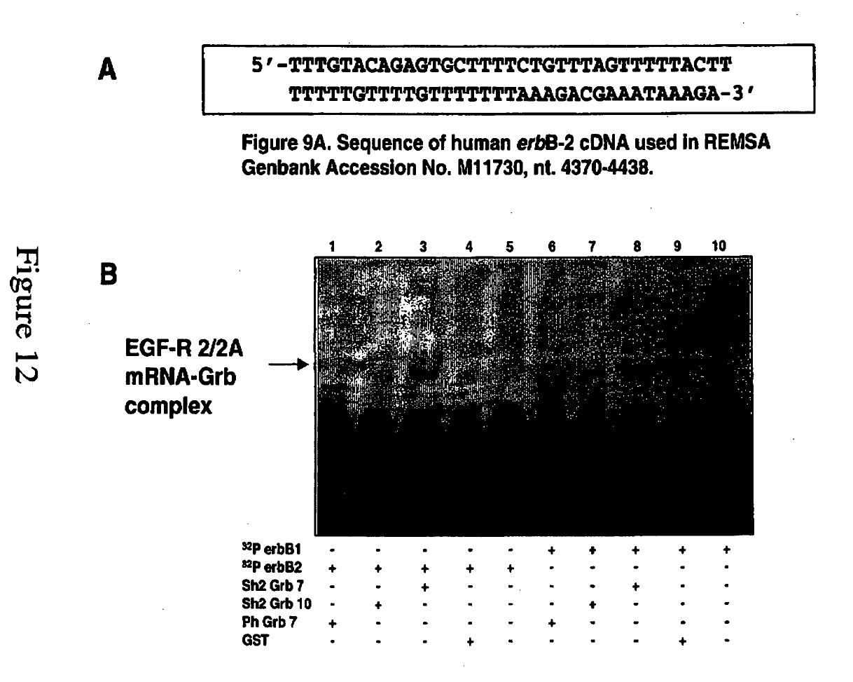

In addition, we generated a longer Grb7 fusion that contained the 3′ end of the protein, and included the Pleckstrin Homology (PH) domain (See FIG. 10A and FIG. 12B). As can be seen, the SH2 domain binds avidly to EGF-R 2/2A mRNA in RNA-gel shift assay (see FIG. 10B). Interestingly, the binding to 2/2A was redox sensitive and the addition of vanadate augmented binding, whilst addition of 2-mercapto-ethanol reduced binding.

Northwestern analysis confirmed binding of GST-Grb7 to ²/_{2A} mRNA (data not shown). In this assay, the GST-Grb7 fusion was resolved on a SDS-PAGE gel, transferred to nitrocellulose and then the membrane probed with a ³²P-labelled ²/_{2A} probe.

For many of the assays, the GST-Grb7 fusion proteins were cleaved with thrombin to generate a smaller ˜13 kD protein. To investigate the specificity of binding by the Grb7, cleaved Grb7 protein was analysed in REMSA with the addition of various unlabeled competitor RNAs in excess. Excess EGF-R2/2A probe competes out formation of the complex completely, whilst excess vector alone has little effect (data not shown). Excess of a probe containing consensus AU-rich nonarner competed well for the complex, consistent with our previous data suggesting that the proteins target AU-rich sequences (data not shown).

### EXAMPLE 6

Mutational Analysis of GRB7 RNA-Binding Domain

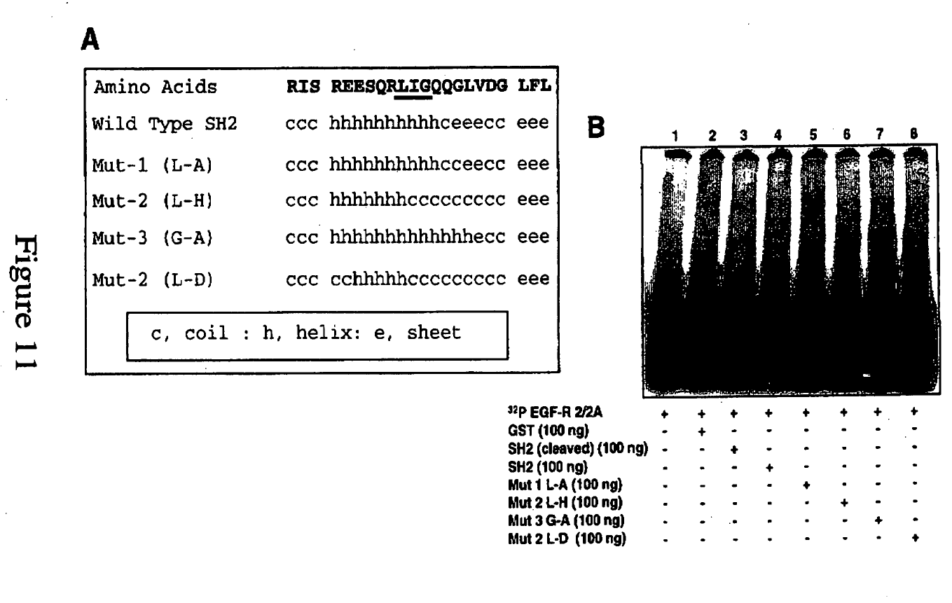

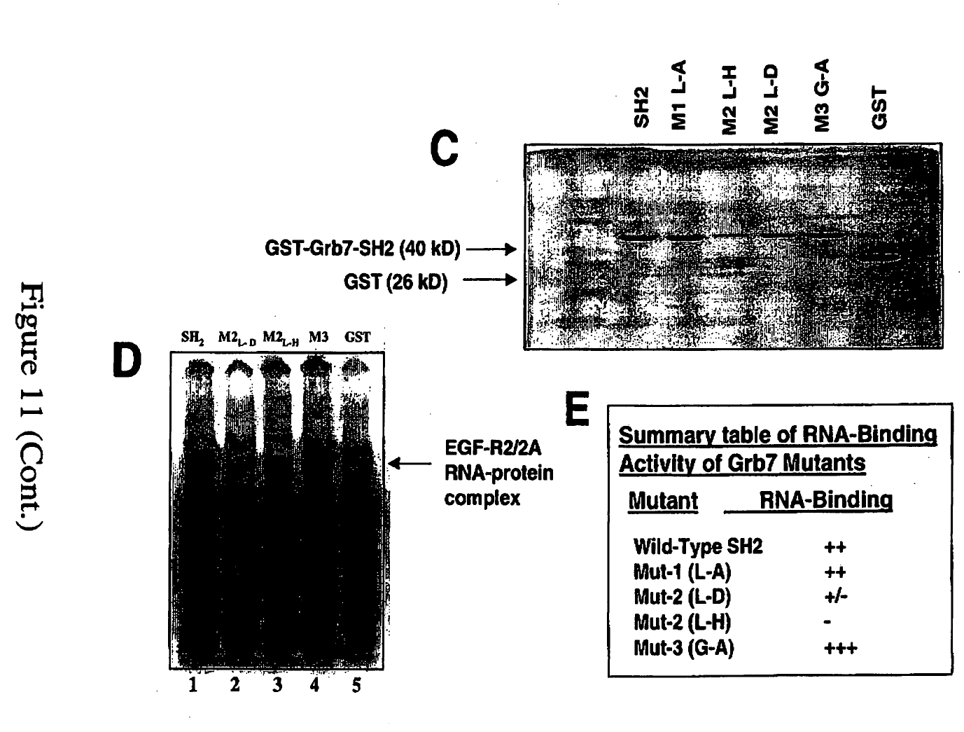

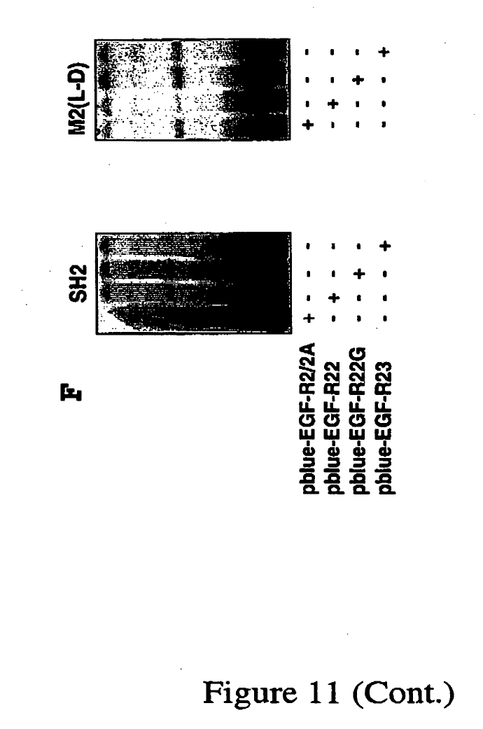

We next modeled the mRNA binding motif (See FIG. 9B) and generated mutants at amino acid residues predicted to modify the a-helix within the KH-like domain. We used the same analysis we had performed to generate the mRNA-binding domain fold pattern, derived from using several secondary structure modelling computer programs (See above). FIG. 11A demonstrates the amino acid sequence of the GST-Grb7 fusion protein mutants that we generated to test the importance of some of the amino acids within the Grb7 mRNA-binding domain. The GST-fusion proteins were generated in the same way as described above. The mutants were generated by standard site directed mutagenesis methods (Maniatis et al., 1990). FIG. 11C shows a Coomassie stain of a SDS-PAGE gel with various GST-Grb7 fusion proteins. The Grb7 mutants showed differential binding to 2/2A (see FIG. 11B and FIG. 11D). These results are summarized in FIG. 11E. For, erbB-1 mRNA, mutations at the leucine (L) residue that reduced the α-helix on secondary structure predictions, resulted in reduced binding by Grb7 (e.g., FIG. 11D, Mut2, L to D, and Mut2, L to H, lanes 2 & 3, respectively). However, mutations that increased the predicted stability of the α-helix (e.g., G to A, Mut3) increased binding (FIG. 11D, lane 4). Thus, the binding capacity of the mRNA binding motif appears dependent upon the integrity of the α-helix.

We next investigated the specificity of cleaved GST-Grb7-SH2 for different mRNA probes within the 2/2A sequence (EGF-R22, 23), and for a mutant within 2/2A that abolishes the 5′ AU-rich sequence (EGF-R22G) (See FIG. 3C). As can be seen in FIG. 11F, neither EGF-R 22 or 23 bind as well to Grb7-SH2 as does the longer EGF-R2/2A sequence. Interestingly, EGF-R22G binds to Grb7-SH2 more avidly than EGF-R22, suggesting that the U-rich sequence is not a major component of the binding site (also consistent with our earlier data, See FIG. 7A and FIG. 7C).

Each of the mutants was also analysed to determine whether alteration in the putative RNA-binding domain would alter RNA specificity. As can been in the panel with Grb7-M1 (See FIG. 11F, M2L-D), the relative binding activity to the EGF-R2/2A and EGF-R22G probes is reversed compared to wild-type Grb7-SH2. This change is maintained for all of the mutants, suggesting that mutations within the RNA-binding domain will contribute significantly to RNA binding specificity.

### EXAMPLE 7

Binding of GRB10 and 14 to ERBB-1 mRNA

Grb10 and Grb14 SH-2 domain fusion proteins also bind to erbB-1 mRNA in RNA gel shift assay. We generated GST-fusion proteins that contained the SH2 domain of other members of the Grb7 family, Grb10 and Grb14 using the same methods as above (see FIG. 10A). The SH2 sequence of human Grb10 included amino acids 520-621 and amino acids 426-540 of human Grb14. The GST-Grb10 and GST-Grb14 fusion proteins were used in REMSA with the EGF-R2/2A probe to determine if they also were able to bind EGF-R mRNA. FIG. 12B shows that Grb10 binds EGF-R2/2A mRNA (lane 7). Binding was also observed for Grb14, although of lower affinity (data not shown). Thus, the ability to bind mRNA is a common property of the Grb7 family of signaling proteins.

### EXAMPLE 8

Binding of the GRB7 Protein Family to ERBB-2 mRNA

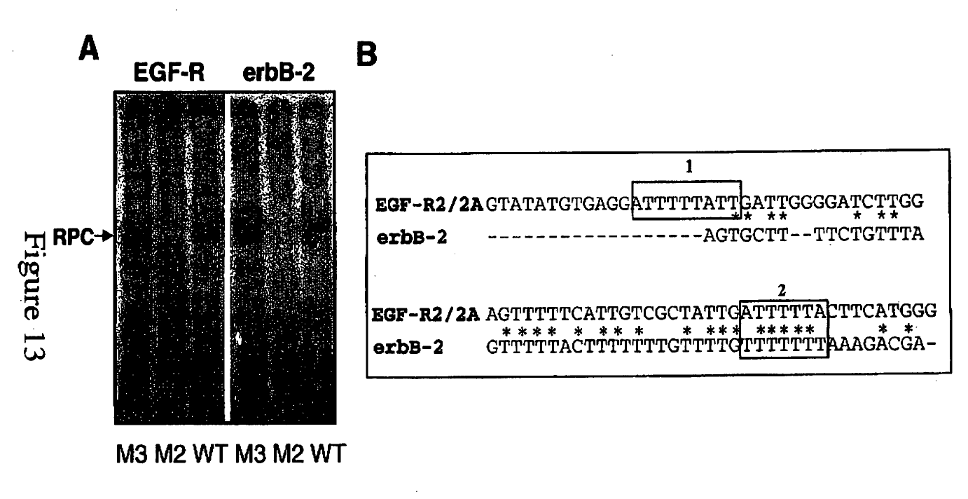

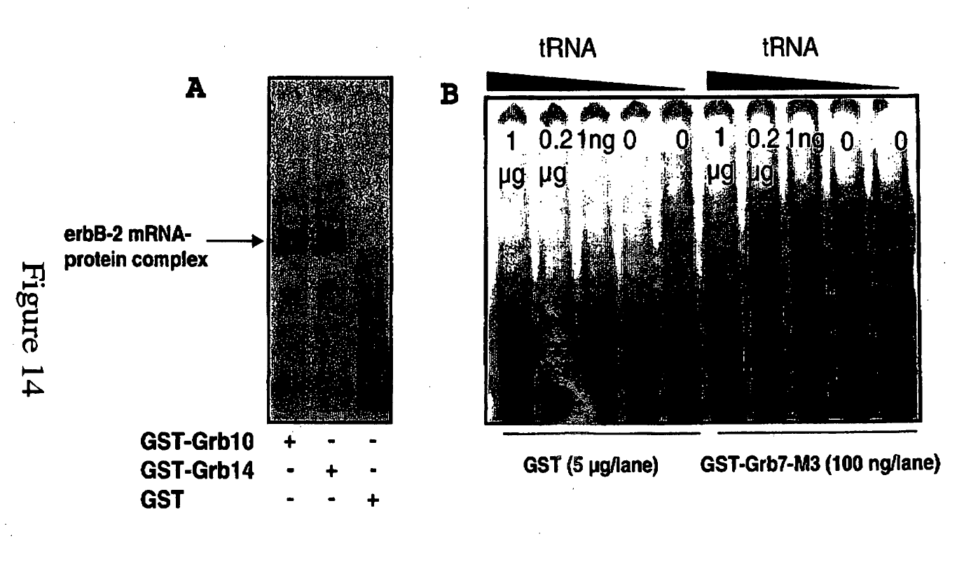

We next investigated the binding of the Grb7 family to erbB-2 mRNA. We used a sequence from the 3′-UTR of erbB-2 that is U-rich and shares homology with other AU-rich sequences (e.g., the c-myc-AU-rich element that has been implicated in destabilization of its mRNA) (FIG. 12A). FIG. 12B shows that Grb7-SH-2 domain wild-type protein binds avidly to erbB-2 mRNA (lane 3). Additional evidence for this is shown in FIG. 13A. Grb7 PH domain, Grb10 and Grb14 also bind to erbB-2 mRNA (See FIG. 12B and FIG. 14A).

We also investigated the binding to erbB-2 mRNA by GST-Grb7 mutants. FIG. 13A shows that the binding to erbB-2 mRNA by two Grb7 mutants (M2 and M3) is similar to the pattern observed for EGF-R2/2A mRNA. That is, reduced binding by the M2 mutant and increased binding by the M3 mutant.

We also investigated the effect of increasing tRNA on the formation of the Grb7-erbB-2 mRNA interaction. FIG. 14B shows that only very large concentrations of tRNA (1 μg) were able to abolish the Grb7-M3-erbB-2 complex. This is consistent with a highly specific interaction.

Taken together, these data demonstrate that the Grb7 family are novel RBPs that can bind to two different growth factor receptor mRNAs, EGF-R (erbB-1) and erbB-2 mRNAs. They do this via a novel KH-like domain within the SH2 domain of the protein. Our data supports a direct role for the KR-like domain in binding RNA. The length of the α-helix within the KH-like domain is a critical determinant of RNA-binding activity. Furthermore, these data suggest that the Grb7 family members bind specific mRNAs with different affinities, reflecting their sequence differences within the core mRNA binding motif.

### EXAMPLE 9

Immunoprecipitation RT-PCR Assay to Detect GRB7-EGF-R RNA Binding in Breast Cancer Cells

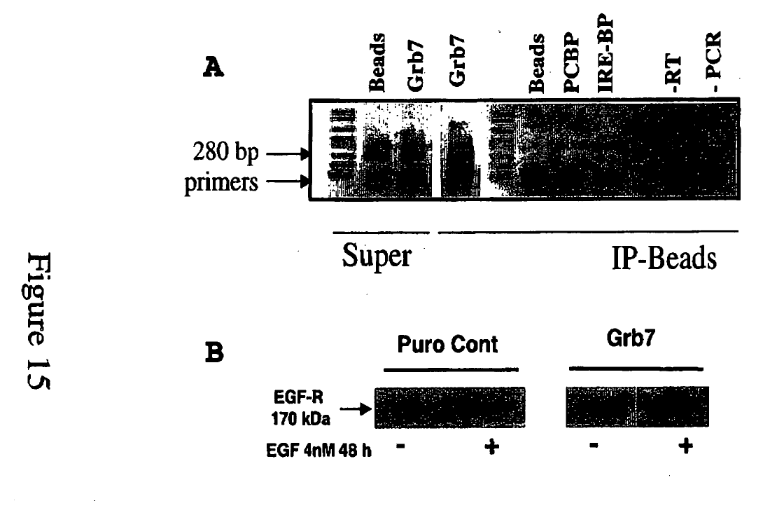

To demonstrate direct binding of Grb7 to EGF-R mRNA in vitro in whole cells, an immunoprecipitation reverse transcriptase polymerase chain reaction (IP-RT-PCR) assay was used (Le et al., 2000). Using this approach with Grb7 antibodies, we were able to IP Grb7 readily from breast cancer cells (data not shown), extract the RNA, generate cDNA and PCR™ up a region that contains the 2/2A sequence of EGF-R mRNA (See FIG. 15A). Human breast cancer cells (SKBR3) were immunoprecipitated with Grb7 or other unrelated antibody (FIG. 15A). RNA was extracted from the immunoprecipitated pellet and supernatant and after being reverse transcribed into cDNA, it was subjected to 40 rounds of PCR™ using two EGF-R primers that amplified a region of 280 nucleotides which included the EGF-R2A sequence used as bait in the original yeast three hybrid screen. The PCR™ products were resolved on agarose gels, and imaged. They showed that Grb7 antibody specifically immunoprecipitated EGF-R mRNA on the beads (FIG. 15A, lane 4), but that other unrelated antibodies (e.g., PolyC-binding protein (PCBP) and iron responsive element-binding protein 1 (IRE-BP)) did not (FIG. 15A, lanes 7 & 8, respectively). RT-PCR™ of the beads alone was negative (FIG. 15A, lane 6). Additional controls for genomic DNA contamination (no RT, lane 9) and cDNA contamination (-PCR™ water control, lane 10) were negative.

Taken together, these data indicate that Grb7 binds directly to EGF-R mRNA in vitro in whole cells, and that the interaction is specific for Grb7. Most importantly, the portion of the EGF-R amplified in the PCR™ covered the bait used in the yeast three hybrid screen used to isolate Grb7 as a novel EGF-R mRNA-binding protein (see above).

### EXAMPLE 10

Over-Expression of GRB7 in Human Breast Cancer Cells

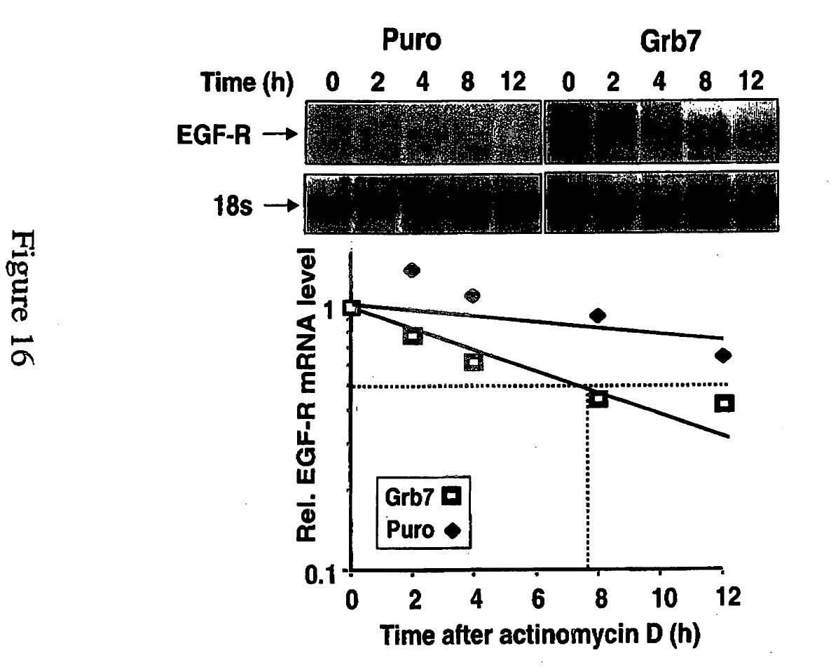

To examine the effects of over-expression of Grb7 in breast cancer cells, Grb7 was cloned into a retroviral expression vector pBabe puro (Morgenstern and Land, 1990) and stably transfected, as described (Pear, Nolan, Scott and Baltimore, 1993) into MDA-468 cells. The stable cell lines were examined using Western blotting and Northern analysis for differences in basal Grb7 protein levels, EGF-stimulated EGF-R levels and basal EGF-R mRNA stability between the puro-vector alone cells and the cells over-expressing Grb7 after actinomycin chase. Results of these experiments are shown in FIG. 15B and FIG. 16. They indicate that overexpression of Grb7 down-regulates EGF-R protein at 48 hr after EGF treatment compared to the upregulation seen in the puro control cells. In addition, FIG. 16 shows that over-expression of Grb7 results in basal destabilization of EGF-R mRNA. This correlates well with the effect seen at the EGF-R protein level, and is consistent with Grb7 acting to destabilize specific mRNAs.

### EXAMPLE 11

Structural Models of the KH-Like RNA-Binding Domain of GRB7

The structure of the KH domain of vigilin was solved in 1996 (Musco et al., 1996). Extensive secondary structure predictions were generated for the KH-like domain of Grb7. Based on a alignment of multiple secondary structure sequence predictions (see above and FIG. 9B), a consensus sequence was identified. We then mapped the amino acid residues within the KH-like domain of Grb7 onto the vigilin structure. The location of the amino acids within the Grb7 KH-like domain predicts that an RNA-binding pocket is formed with the three beta sheets at the floor bracketed by two alpha helices. Based on our structure, we were able to predict the effect on RNA-binding of specific amino acid mutations of the KH-like domain. Moreover, structural prediction of the effect of mutations within the core (LIG) motif based on changes in the secondary structure were readily evident on the predicted 3-dimensional model.

To further define the molecular structure of the SH2 domain of Grb7, a more extensive analysis was performed based on the protein data bank (PDB). The amino acid sequence of Grb7 was subjected to a BLAST search against the PDB, and the highest match was with Syk kinase SH2 domain (PDB number la81) (Futterer et al., 1998). The structure of Syk kinase SH2 domain had been solved by crystallography. A structural alignment was performed with Syk which enabled a consensus structure to be built, and a tertiary structure to be predicted.

General Methods and Procedures

RNA Isolation and Northern Analysis

Cells were solubilized in 4M guanidinium isothiocyanate and total RNA was isolated using the method of Chomczyniski and Sacchi (1987). RNA (10-15 μg per sample) was size fractioned on a 1% agarose-formaldehyde gel and transferred to Hybond-N+ membrane (Amersham, Australia). RNA was UV cross-linked to the membrane, which was prehybridized for 4 h at 42° C. in a buffer containing 50% formamide, 0.75 M NaCl, 0.075 M Na citrate pH=7.0, 5× Denhardt's solution, 1% SDS and 200 μg/mL salmon sperm DNA and then hybridized in the same buffer overnight at 42 C with ³²P-labeled EGF-R cDNA probe at 10⁶ cpm/mL. The membrane was washed with 2×SSC containing 0.1% SDS, then 0.2×SSC containing 0.1% SDS at 22° C. Membranes were analyzed by autoradiography using Kodak EM-1 film, imaged with a PhosphorImager™ and quantitated using ImageQuant™ software. In all experiments an 18S ribosomal RNA probe was used for normalization.

mRNA Turnover Studies

Cells were grown to 70-80% confluency and treated with EGF (25 ng/mL, 4 nM) (Promega, Madison, USA), for 8 h followed by the addition of the transcription inhibitor actinomycin D (Act D) at 7.5 μg/mL (Sigma, St. Louis, Mo.). Total RNA was isolated from the cells at 0, 2, 4, 8 and 12 h time intervals after addition of actinomycin D and subjected to Northern analysis as described earlier by McCulloch et al., 1998. EGF-R mRNA half-life was determined using linear regression analysis.

Immunoblot Assay for EGF-R Protein

Cells were treated with EGF for 8 h, harvested and lysed in ice-cold lysis buffer [1% Triton X-100®, 20 mM Tris-HCl (pH 7.4), 1 mM EDTA], as described by Seth et al. (1999). After 10 min. on ice, the lysate was centrifuged at 750× g (Eppendorf centrifuge 5415C) for 10 min. at 4° C., after which the supernatant was recovered, and stored at −85° C. Total protein concentrations of lysate were determined using the BioRad protein assay, and the protein lysate (5-10 μg/lane) was electrophoresed on 6% SDS-PAGE gels and transferred to nitrocellulose membranes. The membranes were blocked with 5% non fat dried milk in TBS-T [20 mM Tris-HCl, pH=7.4, 150 mM NaCl, 0.1% Tween-20®] at 22° C. for 1 h, prior to incubation with an EGF-R polyclonal antibody (UpState Biotechnology, Lake Placid, N.Y.) (1:2,000) for 1-2 h at 22° C. This was followed by incubation with horseradish peroxidase-conjugated anti-sheep goat IgG (1:2,000) (Amersham, United Kingdom), and the EGF-R protein was visualized by enhanced chemiluminescence (Amersham, United Kingdom) and autoradiography. The 170-kD EGF-R protein bands were quantitated using a Kodak Digital DCS-420c camera and ImageQuant™ software.

Transfection, Luciferase and β-Galactosidase (β-Gal) and RT-PCR Assays

Cells (70-80% confluent), were transfected by electroporation with 10 μg RSV-Luc or RSV-Luc/EGF-R and 6 μg of RSV-β-gal as control. After electroporation, cells were cultured in medium in the presence or absence of EGF (25 ng/mL) for 6 h prior to lysate extraction and assays for Luc and β-gal activity. Lysates were prepared by harvesting the cells from the plate in phosphate buffered saline (PBS). The mixture was centrifuged at 450×g (Jouan C3-12) for 5 min., the supernatant removed, and the cell pellet resuspended in 250 μl lysis buffer (125 mM Tris pH=7.6, 0.5% Triton X-100@). The solution was centrifuged at 16500×g (Eppendorf centrifuge 5415C) for 10 min. at 4° C., and the supernatant used in the Luc assay. Fifty μl of the lysate was used in each assay with 250 μl of assay buffer (25 mM glycylglycine pH=7.8, 15 mM MgSO₄). Samples in triplicate were analyzed in an automated Berthold Luminometer, with 100 μl of luciferin mixture (containing luciferin (50 μg/mL) (Promega Corp., Madison, Wis., USA), 5 mM ATP and assay buffer) added to each sample. β-gal activity was determined for each extract as described (Rosenthal, 1987).

Cell-Free mRNA Decay Assay

To independently assess whether the EGF-R mRNA 3′-UTR cis-acting elements in EGF-R1A and EGF-R2A were stabilized by EGF, we used a cell-free mRNA decay system (Ross, 1994) Briefly, ³²P-labeled EGF-R1A and EGF-R2A riboprobes were transcribed and incubated at 37 C with polysomes extracted from MDA-MB-468 cells that had been grown in the presence or absence of EGF. Aliquots were removed from the reaction mixture at various time points and RNA extracted, followed by electrophoresis on a formaldehyde gel prior to transferring to a nylon membrane and imaging by PhosphorImager™. RNA decay rates were determined by quantitation of the remaining riboprobe.

Preparation of Cytoplasmic Extracts

MDA-MB-468 cells were grown to 70 to 80% confluency in 100 mm culture dishes. Cells were washed with phosphate-buffered saline (PBS) and media replenished 12 to 24 h prior to ligand treatment with EGF (25 ng/mL) or 4-beta-phorbol 12-myristate 13-acetate (PMA) (50 ng/mL). Cells were scraped from the culture dishes with chilled PBS, centrifuged for 4 min. 450×g (Jouan centrifuge C3-12) at 4 C, the supernatant removed, the cells washed again with PBS and centrifuged at 450×g (Jouan centrifuge C3-12) for 4 min. Cell pellets were incubated with cytoplasmic extract buffer (10 nM HEPES, 3 mM MgCl₂, 40 mM KCl, 5% glycerol, 0.2% NP-40™, 1 mM DTT, with freshly added protease inhibitors (0.5 mM phenylmethlsulfonyl fluoride (PMSF), 10 μg/mL leupeptin, 2 μg/mL aprotonin [Sigma Chemical, St. Louis, Mo., USA]), for 20 min. followed by centrifugation for 2 min. at 12,100×g (Eppendorf centrifuge 5415C), and 4° C., and the supernatant snap frozen in liquid nitrogen. Extracts from MCF7, A431, SKBR-3, LNCaP and DU-145 cells were all processed in a similar manner. Protein concentrations were determined using the by BioRad protein assay kit.

RNA Electrophoretic Mobility Shift Assay (REMSA)

Binding reactions were performed as described (Thomson et al., 1999) with 5 μg of cytoplasmic extract and 10⁵ cpm of RNA (˜2-5 pg). Briefly, following incubation at 22° C. for 30 min., 0.3 U of RNase T1 (Roche, Indianapolis, Ind.) was added for 10 min., followed by the addition of heparin (final concentration 50 mg/mL) (Sigma, St. Louis) for 10 min. Samples were subjected to electrophoresis on a 4% native acrylamide gel (acrylamide/bisacrylamide ratio 36:1), dried and analyzed by PhosphorImager™ followed by autoradiography.

In some assays, extracts were preincubated with sense unlabeled RNA (50-150 fold excess) prior to the addition of ³²P-riboprobes. Unlabeled competitor was denatured at 70° C. for 10 min., renatured at 22° C. for 15 min., combined with 5 μg protein cell extract for 30 min. before the addition of ³²P-riboprobes for 30 min., followed by RNase T1, heparin and electrophoresis as described above. In some assays, antibodies to specific RNA-binding proteins were added (as described, Thomson et al., 1999) in an effort to supershift RNA-protein complexes.

UV Cross-Linking of RNA Protein Complexes

RNA-protein binding reactions were carried out as described above using 20-30 μg of cytoplasmic extract and 1.5×10⁵ cpm of RNA (10-15 pg) of ³²P-riboprobe (Thomson et al., 1999). Following the addition of heparin, samples were placed on ice in a microtitre tray and UV-irradiated for 10-15 min. 1 cm below the Stratalinker™ UV light source (Stratagene, 240 nm UV-bulb, La Jolla, Calif.). Samples were incubated after UV cross-linking with RNase A (final concentration, 100 μg/ML) (Roche, Australia) at 37° C. for 15 min. The samples were boiled for 3 min. in SDS sample buffer, subjected to 8-10% SDS-polyacrylamide gel electrophoresis, and analyzed by autoradiography. ¹⁴C molecular weight markers (Amersham), along with prestained Rainbow markers (Amersham), were used as standards.

Yeast 3-Hybrid Screening

The procedure used was according to SenGupta et al., 1996. The EGF-R 2/2A sequence was subcloned into the pIIIA/MS2-1 vector using SmaI digestion, creating the pIIIA-2/2A-MS2-1 plasmid, and the plasmid sequence verified by dideoxy sequencing. The RNA hybrid plasmid was introduced into the yeast strain L40 coat and selected on Trytophan- (Trp-) and uracil- (Ura-) plates. The breast cancer activation domain cDNA library was then introduced into the L40 strain containing pIIIA-MS2-2/2A, and selected using Leucine- (Leu-) and Histidine- (His-) plates. White colonies are positive, red colonies negative. 30,000 clones screened. The white colonies were rescreened on LH- plates to confirm true positives.

The interaction was tested on LH- plates with 3-aminotriazole (˜10-20 mM 3AT) to confirm positives as 3AT reduces background and false positives. Further selection was performed using 5-fluroorotic acid, which detects non-specific protein-protein interactions and ensures that positives require the RNA-protein interaction. Plasmid DNA was isolated from the yeast positive, transformed into DHSalpha bacterial cells and sequenced. One clone contained the 3′ 1044 nucleotides of the human Grb7 cDNA. The entire clone was sequenced from both ends.

Generation of GST-Fusion Proteins

The Grb7 (amino acids 415-532), Grb10 (amino acids 520-621) and Grb14 (amino acids 426-540) SH2 domains were subcloned into the BamHI site of the pGEX4T2 vector and large scale DNA stocks prepared using Qiagen kits. To generate fusion protein, a single colony from a LB ampicillin (Amp) plate was grown overnight at 37° C. as a 50-mL culture. The following morning, the 50 mL culture was used to inoculate 1 L of pre-warmed LB/Amp and grown until the OD.=˜0.6. Then IPTG (0.2 mM) was added and the culture incubated at 37° C. for 4 h. the cells were then centrifuged at 4° C., 3000 rpm for 10 min, before resuspension of the bacterial pellet in TEG (25 mm Tris pH=7.8, 250 mM sucrose, 2 mM EDTA) containing 1 mg/mL lysozyme, 0.5 mM PMSF, 1 μg/mL aprotinin, 1 μg/mL leupeptin, and incubation on ice for 30 min. The pellet was lysed with 0.2% Triton X-100@ and needle lysis before centrifugation at 8,000 rpm for 15 min at 4° C. The supernatant was added to pre-swelled glutathione beads (Sigma) with 1 mM DTT and protease inhibitors and incubated overnight at 4° C. on a rotator. The lysate was centrifuged the next day at 2,000 rpm for 2 min, the beads washed 3 times with TEG buffer before elution with 50 μl of 250 mM reduced glutathione in Tris HCl, pH=8, containing protease inhibitors and DTT as above. After centrifugation at 10,000 rpm for 2 min at 4° C., the supernatant was collected and protein concentration determined using the BioRad Bradford assay.

Retroviral Infection

The full-length Grb7 cDNA was cloned into the retroviral vector pBabe puro (Morgenstern and Land, 1990) and purified by Qiagen maxi-preparation. The construct was then transiently transfected into the retroviral packaging cell line BING (Pear et al., 1993) using Fugene® or calcium phosphate. Briefly, 40 μl of Fugene 6® transfection reagent (Boehringer Mannheim) was added directly into a tube containing 460 μl of serum free media and incubated at room temperature for 5 min.

The diluted Fugene 6® reagent was then added dropwise into a second tube containing 20 μg of pBabe puro-Grb-7 or vector alone. The tube was gently tapped to mix the contents and then incubated at room temperature for 15 min before being added dropwise onto the plates containing BING cells in 10 ml of media. BING cells were grown to ˜65% confluence on 10-cm plates in DMEM plus 10% FCS. Retroviral containing conditioned media was collected from the BING cells at ˜48 h after transfection. Following filtration (0.45 μm) and the addition of 4 μg/mL Polybrene®, the retroviral containing media was added to the target cells (MDA-MB-468) and left overnight. Cells were selected in 1 μg/mL puromycin (Sigma) starting 48 h after infection. Pools of puromycin resistant cells were analysed by Western blotting of Grb7 protein to confirm transgene expression. This showed an ˜15-20-fold increase in Grb7 protein in the stably expressing cell lines. All subsequent experiments were performed using pools of infected cells. Cells simultaneously infected with pBabe puro vector alone served as a control.

Immunoprecipitation Reverse Transcriptase Polymerase Chain Reaction (IP-RT-PCR) Assay

SKBR3 breast cancer cells were grown to ˜70% confluency. EGF was added (4 nM) and the cells incubated for 2 hr before media was removed, the cells washed with ice-cold Phosphate Buffered Saline (PBS). 1 ml of RIPA buffer (1% NP-40, 0.1% SDS, 0.5% Na deoxycholate, 150 mM NaCl, 50 mM Tris HCl(PH=8.0)) was added to the cells for 60 min at 4° C. The lysate was removed and centrifuged at 500 rpm for 10 min, before the supernatant was transferred to a fresh tube. Specific antibodies (Abs) were added: 4 μg Grb7 Ab: N-20, C-20 (Santa Cruz); 4 μg for EGF-receptor Ab: EGFR-Ab-5 (Neomarkers) and incubated at 4° C. for 60 min. Protein A beads (10 mg, Pharmacia) that were pre-swollen and washed ×3 in RIPA buffer 4° C. were incubated with the mixture for 60 min at 4° C. The sample was then spun at 11,500 rpm for 2 min and the supernatant removed to fresh tubes for RNA extraction. The beads were also washed 4 times in RIPA buffer and resuspend in 200 μl DEPC water and extraction of RNA. A portion of the supernatant was also used for Western analysis of immunoprecipitated Grb7 protein.

RNA was extracted from supernatant and beads using phenol(acidic)/chloroform extraction. The RNA was precipitated with 60 μg tRNA, 50 μl (1/10^{th} vol) 3 M Na Acetate (pH 4.2) and 500 μl (0.7 vol) isopropanol at −20° C. for 60 min. The RNA was spun for 15 min at 14,000 rpm. The pellet was washed in 70% alcohol and resuspend in 10 μl DEPC water.

The following protocol was used for the RT-PCR from the RNA.

RNA (1 μl) was added to a primer (oligo dT, random hexamer and reverse primer) together with 9 μl of pure water and incubated at 80° C. for 10 min, before transfer to ice.

The following were added to a final volume of 20 μl:

4 μl first strand buffer (GIBCO)

| | | | |
|---|---|---|---|
| DTT | 2 μl | 0.1 | M |
| DNTPs | 1 μl | 10 | mM |

Superscript II™ (GIBCO) (200 U)

The mixture was incubated at 25° C. for 10 min; 42° C. for 55 min; and then 70° C. for 15 min.

The mixture was then subjected to PCR™. The following were combined into one tube:

| cDNA | | 2.0 | μl |
|---|---|---|---|
| 50 ng μl⁻¹ | Primer 1 | (1.0 | μl) |
| 50 ng μl⁻¹ | Primer 2 | (1.0 | μl) |

| | | |
|---|---|---|
| 2 mM dNTP's | (2.0 | μl) |
| 50 mM MgCl₂ | (0.6 | μl) (1.5 mM final conc) |
| X10 buffer | (2.0 | μl) |
| Taq DNA polymerase | (0.2 | μl) (1 U GIBCO) |
| MilliQ ™ water | (12.2 | μl) |
| Total volume | (20.0 | μl) |

Each sample was subjected to the following rounds of PCR™ in a Perkin Elmer Thermal Cycler:

| | | | |
|---|---|---|---|
| 1 X | 95° C. 5′ | 55° C. 1′ | 72° C. 1′ |
| 40 X | 95° C. 1′ | 55° C. 1′ | 72° C. 1′ |
| 1 X | 95° C. 1′ | 55° C. 1′ | 72° C. 10′ |

At the completion of the reaction, each sample was resolved on 1.5% agarose TAE gels.

Molecular Modeling of GRB7 KH-Like Domain

Two models were generated:

In the first, secondary structure predictions were generated, a consensus sequence identified and then mapped onto the solved structure of vigilin (Musco et al., 1996). The resulting structure shares many features of the vigilin backbone, but diverges in the 3′ end of the KH-like domain.

In the second model, the Grb7 SH2 domain sequence was searched against the known solved crystallographic structures in the PDB. Syk kinase was the highest match (PDB No 1a81). Using the Syk structure (Futterer et al., 1998), a structural alignment was performed using Structural Neighbours program (through http://pdb.bic.nus.edu.sg) and CE (combinatorial extension of the optimal path) and FSSP (Fold classification based on structure-structure alignment of proteins). Both of these latter programs were obtained through (tp://xvw.pdb.bic.nis.edu.sg/pdb/cpilexpore). The best fit/top matches were selected for alignment with Syk, and loaded into Swiss-PDB viewer to build a consensus structure. The Grb7 sequence was aligned and a tertiary structure was predicted. The program WhatCheck was used (at swissmod@ggr.co.uk) for checking the structural prediction, and CNS (crystallography and NMR system) used for energy minimisation. Additional modifications were made to the structure with the program ONO.

References

U.S. Pat. No. 4,376,110

U.S. Pat. No. 4,816,567.

Balmer, Beveridge, Jazayeri, Hollams, Thomson, Walker and Leedman, “Identification of a novel AU-rich element in the 3′-untranslated region of EGF-receptor mRNA that is the target for regulated RNA-binding proteins,” Mol. Cell Biol., revision resubmitted, 2000.

Barlat et al., “A role for Sam68 in cell cycle antagonized by a spliced varaint within the KH domain,” J. Biol. Chem., 272:3129-32, 1997.

Batzer et al., “Heirachy of binding sites for Grb2 and Shc on the EGF-receptor,” Mol. Cell Biol., 14:5192-201, 1994.

Bayer et al., Meth. Enz., 184:138-63, 1990.

Brodeur et al., MONOCLONAL ANTIBODY PRODUCTION TECHNIQUES AND APPLICATIONS, Marcel Dekker, Inc., New York, pp. 51-63, 1987.

Brown et al., Meth. Enzymol., 68:109-51, 1979.

Bruggermann et al., Year in Immuno., 7:33, 1993.

Carraway et al., “The erb133 gene product is a receptor for heregulin,” J. Biol. Chem., 269:14303-06, 1994,.

Carter, Meth. Enz., 154:382-403, 1987.

Caruther et al., Meth. Enzymol., 154:287-313, 1985.

Chen and Shyu, “AU-rich elements: characterisation and importance in mRNA degradation,” TIBS, 20:465-70, 1995.

Chomczynski and Sacchi, “Single-step method of RNA isolation by acid quanidinium thiocyanate-phenol-chloroform extraction,” Anal. Biochem., 162:156-59, 1987.

Craik et al., Science, 228:291-97, 1985.

Daly et al., “Cloning and characterisation of GRB.14, a novel member of the GRB7 gene family,” J. Biol. Chem., 271:12502-10, 1996.

David et al., Biochemistry, 13:1014-21, 1974.

David et al., “STAT activation by epidermal growth factor (EGF) and amphiregulin,” J. Biol. Chem., 271:9185-88, 1996.

Davis, “The mitogen-activated protein kinase signal transduction pathway,” J. Biol. Chem., 268:14553-56, 1993.

Engels et al., Agnew. Chem. Int. Ed. Engl., 28:716-34, 1989.

Fernandez-Pol, “Modulation of EGF receptor protooncogene expression by growth factors and hormones in human breast carcinoma cells,” Critical Rev. Oncogenesis, 2:174-85, 1991.

Filmus et al., “MD4-468, a human breast cancer cell line with a high number of epidermal growth factor (EGF) receptors, has an amplified EGF receptor gene and is growth inhibited by EGF,” Biochem. Biophys. Res. Commun., 128: 898-905, 1985.

Frohman, et al., Proc. Nat. Acad. Sci. USA, 85:8998-9002, 1988.

Fugamalli et al., “A target for Src in mitosis,” Nature, 368: 871-74, 1994.

Futterer, Wong, Grucza, et al., “Structural basis for Syk tyroinse kinase ubiquity in signal transduction pathways revealed by the crystal structure of its regulatory SH2 domains bound to a dually phosphorylated ITAM petide,” J. Mol. Biol., 281:523-28, 1998.

Greenberg and Belasco, “Control of the degradation of labelled protooncogene and lymphokine mRNAs,” In CONTROL OF mRNA STABILITY, Belasco and Brawerman (eds.), Academic Press, pp. 199-218, 1993.

Higuchi, In PCR PROTOCOLS, Academic Press, pp. 177-83, 1990.

Hobert et al., “Novel signalling pathway suggested by SF13 domain-mediated p95vav/Heterogenous Ribonucleoprotein K interaction,” J. Biol. Chem., 269:20225-28, 1994.

Holmes et al., “Identification of heregulin, a specific activator of p185erbB2,” Science, 256:1205-08, 1992.

Hoogenboom et al., J. Mol. Biol., 227:381, 1991.

Horwitz et al., Meth. Enz., 185:599-611, 1990.

Jakobovits et al., Proc. Natl. Acad Sci., 90:2551-55, 1993.

Jakobovits et al., Nature, 362:255-58, 1993.

Jones et al., Nature, 321:522-25, 1986.

Keller et al., in DNA PROBES, Stockton Press, pp. 11-18, 1989.

Kennedy et al., “A novel mouse protein (G313P) containing SH3 domain-binding consensus sequences and RNA-binding domains implicates a new family of signal transduction proteins,” Submitted, 1998.

Kennedy, Wood, Ramsdale, Tam, Steiner and Mattick, “Identification of a mouse orthologue of the human Ras-GAP-SH3-domain binding protein and structural confirmation that these proteins contain an RNA recognition motif. Biomedical peptides, proteins & nucleic acids,” 2:93-99, 1997.

Klijn et al., “Endocrine aspects of cancer,” Endocrin. Rev. Monographs, 1:171-74, 1993.

Kohler et al., Nature, 256:495-97, 1975.

Kozbor, J. Immunol., 133:3001, 1984.

Lagnado et al., “AUUUA is not sufficient to promote poly(A) tail shortening and degradation of an mRNA: the functional sequence within AU-rich elements may be UUAUUUA(U/A)(U/A),” Mol. Cell Biol., 14:7984-95, 1994,.

Le, Sternglanz and Greider, “Identification of two RNA-binding proteins associated with human telomerase RNA,” Mol. Biol. Cell, 11:999-1010, 2000.

Margolis et al., “High-efficiency expression/cloning of epidermal growth factor-receptor-binding proteins with Src homology 2 domains,” Proc. Natl. Acad. Sci. USA, 89:8894-98, 1992.

Marks et al., J. Mol. Biol., 222:581, 1991.

McCulloch, Walker, Chakera, Jazayeri and Leedman, “Regulation of EGF-receptor expression by EGF and TGFα in epidermoid cancer cells is cell type-specific,” Int. J Biochem. Cell Biol., 30:1265-78, 1988.

Merlino et al., “Enhanced expression of the EGF-receptor gene in A431 human carcinoma cells,” Science, 224:417-19, 1984.

Morgenstern and Land, “Advanced mammalian gene transfer: high titre retroviral vectors with multiple drug selection markers and a complementary helper-free packaging cell line,” Nucleic Acids Res., 18:3587-96, 1990.

Morrison et al., Proc. Natl. Acad. Sci., 81:6851-55, 1984.

Mullis et al., Meth. Enzymol., 155:335-50, 1987.

Musco et al. “3-D structure and stability of the KH domain: molecular insights into the fragile X syndrome,” Cel, 85:237-45, 1996.

Nakagawa et al., “AUH—a gene encoding an AU-specific RNA-binding protein with enoyl-CoA intrinsic hydratase activity,” Proc. Natl. Acad. Sci. USA, 92:2051-55, 1995.

Narang et al., Meth. Enzymol., 68:90-98, 1979.

Ochman et al., in PCR™ PROTOCOLS, Academic Press, pp. 219-27, 1990.

Ooi et al., “The cloning of Grb10 reveals a new family of SH2 domain proteins,” Oncogene, 10:1621-30, 1995.

Pain et al., J. Immunol. Meth., 40:219-31, 1981.

Pawson, “Protein modules and signalling networks,” Nature, 373:573-80, 1995.

Pear, Nolan, Scott and Baltimore, “Production of high-titer helper-free retroviruses by transient transfection,” Proc. Natl. Acad Sci. USA, 90:8392-96, 1993.

Peltz et al., “Regulation of mRNA turnover in eukaryotic cells,” Crit. Rev. in Eukaryotic Gene Express, 1:99-126, 1991.

Perry et al., Science, 226:555-57, 1984.

Pinol-Roma and Dreyfuss, “Shuttling of pre-mRNA binding proteins between the nucleus and cytoplasm,” Nature, 355:730-32, 1992.

Rhee and Choi, “Regulation of inositol phospholipid-specific phospholipase C isoenzymes,” J. Biol Chem., 267:12393-96, 1992.

Riechmann et al., Nature, 332:323-27, 1988.

Rosenthal, “Identification of regulatory elements of cloned genes with functional assays,” Methods Enzymol., 152:704-20, 1987.

Ross, “Analysis of mRNA turnover in cell free extracts from mammalian cells,” In RNA PROCESSING: A PRACTICAL APPROACH, Vol II., Higgins and Hames (eds.), 1994.

Sagesser et al., “Detection and isolation of RNA-binding proteins by RNA-ligand screening of a cDNA expression library,” Nuc. Acids Res., 28:1045-52, 2000.

Saiki et al., Science, 239:487-92, 1988.

Salamon et al., “Studies of the HER-2/neu protooncogene in human breast and ovarian cancer,” Science, 244:707-11, 1989.

Salamon et al., “Loss of growth responsiveness to epidermal growth factor and enhanced production of alpha-transforming growth factors in ras transformed mouse mammary epithelial cells,” J. Cell Physiol., 130:397-406, 1987.

Sambrook et al., MOLECULAR CLONING, Cold Spring Harbor Laboratory Press, pp. 1.74-1.84 and 16.30-16.55, 1989.

Sandgren et al., “Over-expression of TGFα in transgenic mice: induction of epithelial hyperplasia, pancreatic metaplasia and carcinoma of the breast,” Cell, 61:1121-35, 1990.

SenGupta et al., “A three-hybrid system to detect RNA-protein interactions in vivo,” Proc. Natl. Acad. Sci. USA, 93:8496-501, 1996.

Seth, Shaw, Jazayeri and Leedman, “Complex post-transcriptional regulation of EGF-receptor expression by EGF and TGFα in human prostate cancer cells,” Br. J. Cancer, 80:657-69, 1999.

Shaw and Kamen, “A conserved Au sequence from the 3′ untranslated region of GM-CSF mRNA mediates selective mRNA degradation,” Cell, 46:659-67, 1986.

Stein et al., “The SH2 domain protein GRB-7 is co-amplified, over-expressed and in a tight complex with HER2 in breast cancer,” EMBO J., 13:1331-40, 1994.

Tanaka et al., “Co-expression of Grb7 with epidermal growth factor receptor or Her2/erb132 in human advanced esophageal carcinoma,” Cancer Res., 57:28-31, 1997.

Thomson, Rogers, Walker, Staton and Leedman, “Optimized RNA gel-shift and UV cross-linking assays for characterization of cytoplasmic RNA-protein interactions,” Biotechniques, 27:1032-42, 1999.

Triglia et al., Nuc. Acids Res., 16:8186, 1988.

Ullrich and Schlessinger, “Signal transduction by receptors with tyrosine kinase activity,” Cell, 61:205-10, 1990.

Vallette et al., Nuc. Acids Res., 17:723-33, 1989.

van Bergen en Henegouwen et al., “The EGF-receptor is associated with actin filaments,” Exp. Cell Res., 199:90-97, 1992.

Verhoeyen et al., Science, 239:1534-36, 1988.

Wang et al., in PCR™ PROTOCOLS, Academic Press, pp. 70-75, 1990.

Wells et al., Gene, 34:315-23, 1985.

Wong et al., “Molecular cloning and nucleic acid binding properties of the GAP Associated Tyrosine Phosphoprotein p62,” Cell, 69:551-58, 1992.

Yamamoto et al., “Amplification of the EGF receptor gene in squamous carcinoma cell lines,” Cancer Res., 46:414-16, 1986.

Zola, MONOCLONAL ANTIBODIES: A MANUAL OF TECHNIQUES, CRC Press, Inc., pp. 147-158, 1987.

Zoller et al., Meth. Enz., 100:4668-500, 1983.

Zoller et al., Meth. Enz., 154:329-50, 1987.

1. An isolated polynucleotide comprising:
a) the nucleotide sequence of SEQ ID NO:1;
b) a nucleic acid sequence that encodes the polypeptide of SEQ ID NO:2;
c) a nucleic acid molecule that has at least 75% sequence homology to the sequences in a) or b); or
d) a nucleic acid molecule that specifically hybridizes to any one of the sequences in a) or b) under stringent hybridization conditions.

2. An isolated polypeptide comprising an mRNA binding motif that comprises the amino acid sequence of SEQ ID NO:2.

3. An antisense polynucleotide that specifically binds to an mRNA binding motif that comprises the nucleotide sequence of SEQ ID NO:1.

4. The antisense nucleic acid of claim 3, wherein said antisense nucleic acid inhibits the activity of said mRNA binding motif in a host cell transformed with said antisense nucleic acid.

5. The antisense nucleic acid of claim 4, wherein the inhibition of said activity alters the proliferation, differentiation, or viability of said host cell.

6. The antisense nucleic acid of claim 3, wherein said antisense nucleic acid comprises the sequence of SEQ ID NO:3.

7. A method of screening for a ligand, agonist or antagonist that specifically binds to, activates or inhibits the mRNA binding motif of claim 1, said method comprising the steps of: contacting a sample suspected of containing said ligand, agonist, or antagonist with said mRNA binding motif; and detecting either the binding of said ligand, agonist or antagonist to said mRNA binding motif, or the activation or inhibition of said mRNA binding motif.

8. A method for determining the presence of a nucleic acid molecule that encodes the mRNA binding motif of claim 1 in a test sample prepared from cells, tissues, or biological fluids, said method comprising the steps of: contacting said test sample with an isolated DNA that encodes all or a portion of said mRNA binding motif, and determining whether said isolated DNA hybridizes to said nucleic acid molecule in said test sample.

9. An antibody, or biologically active fragment thereof, that specifically binds to the mRNA binding motif of claim 1.

10. The antibody of claim 9, wherein said antibody is an antagonist or an agonist of said mRNA binding motif.

11. A peptide fragment of an mRNA binding motif according to claim 1, capable of eliciting an antibody that co-precipitates a mRNA binding motif ligand.

12. The peptide fragment of claim 11, wherein said fragment comprises the amino acid sequence of SEQ ID NO:2.

13. An antibody elicited by an mRNA binding motif fragment according to claim 12.

14. A polypeptide that is specifically co-precipitated by the antibody of claim 13, from a cell expressing a protein comprising an mRNA binding motif according to claim 13.
